(19) United States
(12) Patent Application Publication
(10) Pub. No.: US 2026/0207150 A1
(43) Pub. Date: Jul. 23, 2026

(54) Stabilization Assembly For A Mobile Medical System

(71) Applicant: Mobius Imaging, LLC, Shirley, MA (US)

(72) Inventor: Russell Stanton, Lunenberg, MA (US)

(73) Assignee: Mobius Imaging, LLC, Shirley, MA (US)

(21) Appl. No.: 19/138,319

(22) PCT Filed: Dec. 12, 2023
(86) PCT No.: PCT/US2023/083536

§ 371 Date: Jun. 12, 2025

Related U.S. Application Data
(60) Provisional application No. 63/431,903, filed on Dec. 12, 2022.

(51) Int. Cl. A61B 6/00; A61B 6/03; A61B 6/04; A61B 6/10

(52) U.S. Cl. CPC A61B 6/4405; A61B 6/035; A61B 6/0407; A61B 6/105; A61B 6/4447; A61B 6/4458

(57) ABSTRACT

A stabilization assembly for providing a mobile medical system with an additional point of contact with a floor surface includes a stabilization housing configured to be coupled to the mobile medical system. The stabilization assembly also includes a foot supported for displacement relative to the stabilization housing between a plurality of foot positions including an extended foot position. The stabilization assembly further includes a biasing element operatively attached to the foot to urge the foot towards the extended foot position. The stabilization assembly also further includes a retainer operable between a released state, and brace state to inhibit movement of the foot away from the floor surface. The retainer is configured to change operation from the released state to the brace state in response to movement of the foot beyond a threshold displacement from the extended foot position occurring in response to abutment of the foot with the floor surface.

### CROSS-REFERENCE TO RELATED APPLICATION

The subject patent application claims priority to and all the benefits of United States Provisional Patent Application No. 63/431,903 filed on Dec. 12, 2022, the disclosure of which is hereby incorporated by reference in its entirety.

### BACKGROUND

Often, mobile medical systems have a propensity to tilt or shift relative to a floor surface on which they are supported due to the floor surface being uneven. This is especially problematic during operation of the mobile medical system. For example, if the mobile medical system is a mobile medical imaging system, tilting or shifting of the system may affect the accuracy of the medical image acquired. Accordingly, there remains a need in the art for addressing one or more of these deficiencies.

### SUMMARY

One general aspect of the present disclosure includes a mobile medical system. The mobile medical system includes a base. The base includes a housing defining a contact surface, one or more wheels, and a base lift interposed between the housing and the one or more wheels. The base lift is operable to move the contact surface relative to a floor surface between a parked mode and a transport mode. In the parked mode, the contact surface abuts the floor surface to inhibit movement of the base along the floor surface. In the transport mode, the contact surface is spaced above the floor surface and with the one or more wheels supporting the base for movement along the floor surface. The mobile medical system also includes a stabilization assembly for providing an additional point of contact with the floor surface in the parked mode. The stabilization assembly includes a stabilization housing coupled to the base. The stabilization assembly also includes a foot extending between a top end and a bottom end and supported for displacement relative to the stabilization housing between a plurality of foot positions. The plurality of foot positions includes an extended foot position where the bottom end is arranged vertically between the contact surface and the floor surface in the transport mode. The stabilization assembly further includes a foot biasing element operatively attached to the foot to urge the foot towards the extended foot position. The stabilization assembly also further includes a retainer operable between a released state to permit movement of the foot relative to the stabilization housing, and a brace state to inhibit movement of the foot away from the floor surface. The retainer is configured to change operation from the released state to the brace state in response to movement of the foot beyond a threshold displacement from the extended foot position occurring in response to abutment of the foot with the floor surface as the base lift moves from the transport mode towards the parked mode.

Another general aspect of the present disclosure includes a mobile medical imaging system. The mobile medical imaging system includes an imaging gantry having at least one imaging component for acquiring image data of a patient. The mobile medical imaging system also includes a base. The base includes a housing supporting the imaging gantry and defining a contact surface, one or more wheels, and a base lift interposed between the housing and the one or more wheels. The base lift is operable to move the contact surface relative to a floor surface between a parked mode and a transport mode. In the parked mode, the contact surface abuts the floor surface to inhibit movement of the base along the floor surface. In the transport mode, the contact surface is spaced above the floor surface and the one or more wheels support the base for movement along the floor surface. The mobile medical imaging system also includes a stabilization assembly for providing an additional point of contact with the floor surface in the parked mode. The stabilization assembly includes: a stabilization housing coupled to the base. The stabilization assembly also includes a foot extending between a top end and a bottom end and supported for displacement relative to the stabilization housing between a plurality of foot positions. The plurality of foot positions includes an extended foot position where the bottom end is arranged vertically between the contact surface and the floor surface in the transport mode. The stabilization assembly further includes a biasing element operatively attached to the foot to urge the foot towards the extended foot position, and a retainer operable between a released state to permit movement of the foot relative to the stabilization housing, and brace state to inhibit movement of the foot away from the floor surface. The retainer is configured to change operation from the released state to the brace state in response to movement of the foot beyond a threshold displacement from the extended foot position occurring in response to abutment of the foot with the floor surface as the base lift moves from the transport mode towards the parked mode.

A further general aspect of the present disclosure includes a stabilization assembly configured to be coupled to a mobile medical system for providing an additional point of contact with a floor surface. The stabilization assembly includes a stabilization housing configured to be coupled to the mobile medical system. The stabilization assembly also includes a foot extending between a top end and a bottom end and supported for displacement relative to the stabilization housing between a plurality of foot positions. The plurality of foot positions includes an extended foot position where the bottom end extends from the stabilization housing at a maximum distance. The stabilization assembly also includes a biasing element operatively attached to the foot to urge the foot towards the extended foot position. The stabilization assembly further includes a retainer operable between a released state to permit movement of the foot relative to the stabilization housing, and brace state to inhibit movement of the foot away from the floor surface. The retainer is configured to change operation from the released state to the brace state in response to movement of the foot beyond a threshold displacement from the extended foot position occurring in response to abutment of the foot with the floor surface.

### BRIEF DESCRIPTION OF THE DRAWINGS

Other advantages of the present invention will be readily appreciated, as the same becomes better understood by reference to the following detailed description when considered in connection with the accompanying drawings.

FIG. 1A is a schematic representation of a mobile medical system disposed on a floor surface in a transport mode and including a stabilization assembly having a foot in an extended foot position.

FIG. 1B is a schematic representation of the mobile medical system disposed on the floor surface in a parked mode and including the stabilization assembly with the foot contacting the floor surface to provide an additional point of contact with the floor surface.

FIG. 2A is a top perspective view of a mobile medical imaging system according to the present disclosure and including a base, a gantry mount in a park pose, and an imaging gantry.

FIG. 2B is a top perspective view of a mobile medical imaging system of FIG. 2A with the gantry mount and an imaging gantry translated along the base in an imaging mode.

FIGS. 3A-3C show various examples of the imaging gantry including at least one imaging component.

FIG. 4 is a schematic representation of the mobile medical imaging system performing a helical scanning procedure.

FIG. 5 is a top perspective view of a schematic representation of the mobile medical imaging system including a gimbal with the imaging gantry tilted relative to the gimbal.

FIGS. 6A and 6B are side and top schematic views, respectively, of the mobile medical imaging system in an imaging mode and the gantry mount in a park pose.

FIGS. 7A and 7B are side and top schematic views, respectively, of the imaging system of FIGS. 6A and 6B in a transport mode and the gantry mount in a transport pose.

FIG. 8 is a representation of an operating room including the mobile medical imaging system and a robotic surgical system.

FIG. 9A-9H show a sequence of side views of the mobile medical system including the stabilization assembly as the base lift operates between a transport mode and a parked mode.

FIG. 10A-10H show a sequence of cross-sectional representations of the stabilization assembly taken through a retainer biasing element to reveal the operation of the stabilization assembly as the base lift operates between a transport mode and a parked mode.

### DETAILED DESCRIPTION

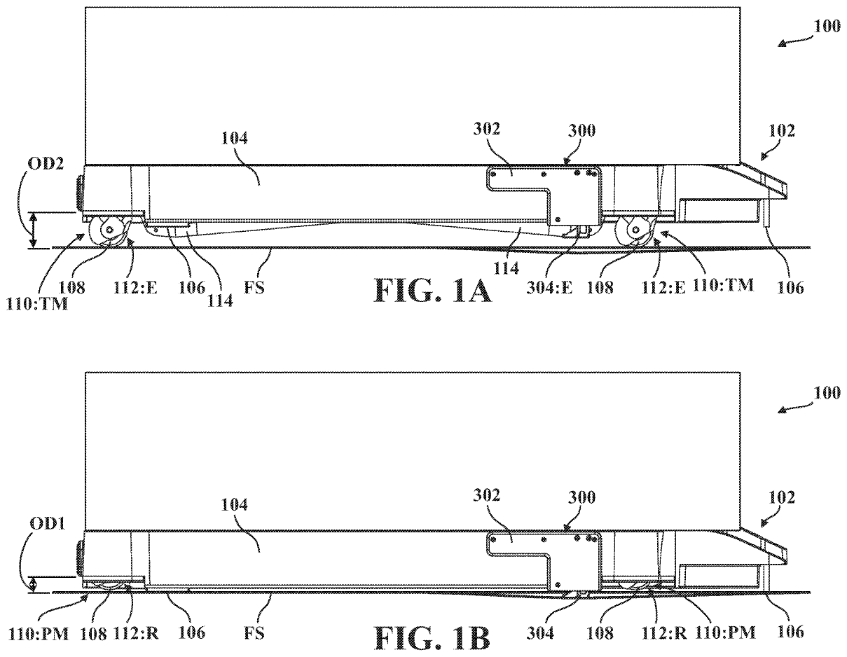

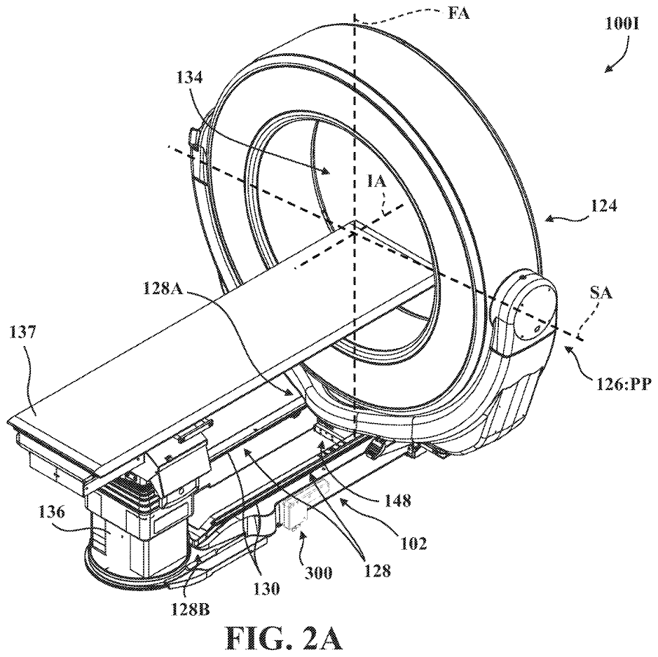

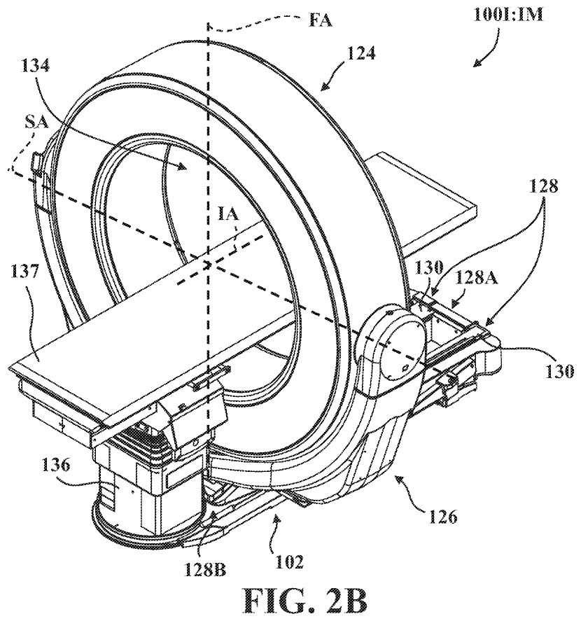

FIGS. 1A-1B generally show a schematic representation of a mobile medical system 100 according to the present disclosure. The mobile medical system 100 may comprise any medical system suitable to be mobile over a floor surface FS. Some examples of mobile medical system 100 include, but are not limited to, carts supporting a robotic arm (such as disclosed in U.S. patent application Ser. No. 11/357,197, Pub. No. US 2006/0142657, filed Feb. 21, 2006, and incorporated by reference herein in its entirety.), a cart supporting a navigation system (such as disclosed in U.S. Pat. No. 7,725,162 issued on May 25, 2010 and U.S. Pat. No. 9,687,307 issued on Jun. 27, 2017, both of which are incorporated by reference herein in their entirety). In other examples, such as shown in FIGS. 2A-2B, the mobile medical system 100 is a mobile medical imaging system 100I (described in greater detail below).

With continued reference to FIGS. 1A and 1B, the mobile medical system 100 generally includes a base 102. The base 102 may include a base housing 104 defining a contact surface 106. In one example, the base housing 104 may have a generally rectangular profile a length and width preferably designed to allow the mobile medical system 100 to fit through most standard-sized doorways (i.e., generally 24-36 inches wide), and to be easily transported through corridors and elevators generally found in hospitals and other healthcare environments. The base 102 further includes one or more wheels 108. The one or more wheels 108 may be operatively attached to the base housing 104 and facilitate movement of the mobile medical system 100 over the floor surface FS. The base 102 further includes a base lift 110 interposed between the base housing 104 and one or more wheels 108. The base lift 110 is operable to move the contact surface 106 of the base 102 relative to the floor surface FS. Particularly, the base lift 110 is operable between a transport mode TM (shown in FIG. 1A) and a parked mode (shown in FIG. 1B). Referring to FIG. 1A, in the transport mode TM, the base lift 110 lifts the base housing 104 relative to the floor surface FS such that the contact surface 106 is spaced above the floor surface FS and the one or more wheels 108 support the base 102 for movement along the floor surface FS. In the parked mode PM, the base lift 110 lowers the base housing 104 such that the contact surface 106 abuts the floor surface FS to inhibit movement of the base 102 along the floor surface FS.

Still referring to FIG. 1A, in one example, the mobile medical system 100 may include one or more casters 112 that act as the base lift 110. The one or more casters 112 each include one of the wheels 108. Each of the one or more casters 112 are supported by a pivoting caster arm assembly 114 interposed between the base 102 and the caster 112. Each pivoting caster arm assembly 114 is configured to pivot relative to the base 102 to move each caster 112 between a retracted position 112R and an extended position 112E. Referring to FIG. 1B, in the retracted position 112R, each caster 112 is spaced from the base 102 at a first offset distance OD1 when the base lift 110 is in the parked mode PM to permit the contact surface 106 to abut the floor surface FS to inhibit movement of the base 102 along the floor surface FS. Referring to FIG. 1A, in the extended position 112E, each caster 112 is spaced from the base 102 at a second offset distance OD2, greater than the first offset distance OD1, when the base lift 110 is in the transport mode TM to lift the base 102 relative to the floor surface FS such that the contact surface 106 is spaced above the floor surface FS and the one or more wheels 108 support the base 102 for movement along the floor surface FS. One exemplary configuration of a base lift 110 including the one or more casters 112 is disclosed in U.S. Patent Application Publication No. U.S. 2022/0061779 entitled “Caster System For Mobile Apparatus,” which is incorporated by reference herein in its entirety. It is also contemplated that the mobile medical system 100 may also include a transport motor that is geared with the one or more wheels 108 to propel the mobile medical system 100 across the floor surface FS. The mobile medical system 100 may also include a steering mechanism for guiding the direction of the mobile medical system 100 over the floor surface FS. Other configurations enabling the base 102 to be mobile relative to the floor surface FS are contemplated.

As described above, for many mobile medical systems 100, it is important for the mobile medical system 100 to be adequately supported on the floor surface FS such that the mobile medical system 100 does not tilt or shift relative to the floor surface FS during operation of the mobile medical system 100. Accordingly, as broadly shown in FIGS. 1A and 1B and described in further detail below, the mobile medical system 100 of the present disclosure further includes a stabilization assembly 300. The stabilization assembly 300 is configured to provide the mobile medical system 100 with an additional point of contact with the floor surface FS when the base lift 110 is in the parked mode PM. By providing an additional point of contact with the floor surface FS, the stabilization assembly 300 ensures that the mobile medical system 100 is in static equilibrium with the floor surface FS such that the mobile medical system 100 does not tilt or shift relative to the floor surface FS during operation of the mobile medical system 100.

In the examples shown in FIGS. 2A-2B, the mobile medical system 100 is a mobile medical imaging system 100I. The mobile medical imaging system 100I may generally include the base 102 (described above) and an imaging gantry 124. The base 102 may define a track 128 extending between a first track end 128A and a second track end 128B. In one example, the track 128 may be defined by a pair of rails 130 that are spaced apart from and parallel to each other. As will be described in further detail below, the mobile medical imaging system 100I may include a gantry mount 126 supports the imaging gantry 124 for movement along the track 128.

The imaging gantry 124 generally includes at least one imaging component 132 and defines an imaging bore 134 defining an imaging axis IA. The mobile medical imaging system 100I is configured to collect imaging data ID, such as, for example x-ray computed tomography (CT) or magnetic resonance imaging (MRI) data, from an object located within the imaging bore 134 of the imaging gantry 124, in any manner known in the medical imaging field. An exemplary imaging gantry 124 that may be used in various versions is the AIRO® intra-operative CT system manufactured by Mobius Imaging, LLC. Examples of x-ray CT imaging devices that may be used according to various versions of the present disclosure are described in U.S. Pat. No. 10,151,810, entitled “Pivoting Multi-directional X-ray Imaging System with a Pair of Diametrically Opposite Vertical Support Columns Tandemly Movable Along a Stationary Base Support;” U.S. Pat. No. 9,962,132, entitled “Multi-directional X-ray Imaging System with Single Support Column;” U.S. Pat. No. 9,801,592, entitled “Caster System for Mobile Apparatus;” U.S. Pat. No. 9,111,379, entitled “Method and System for X-ray CT Imaging;” U.S. Pat. No. 8,118,488, entitled “Mobile Medical Imaging System and Methods;” and U.S. Patent Application Publication No. 2014/0275953, entitled “Mobile X-ray Imaging System,” the disclosures of each of which are hereby incorporated by reference in their entirety. Notably, as shown in FIGS. 2A and 2B, the mobile medical imaging system 100I includes the stabilization assembly 300 to ensure that the mobile medical imaging system does not tilt or shift relative to the floor surface FS during operation of the mobile medical imaging system 100I.

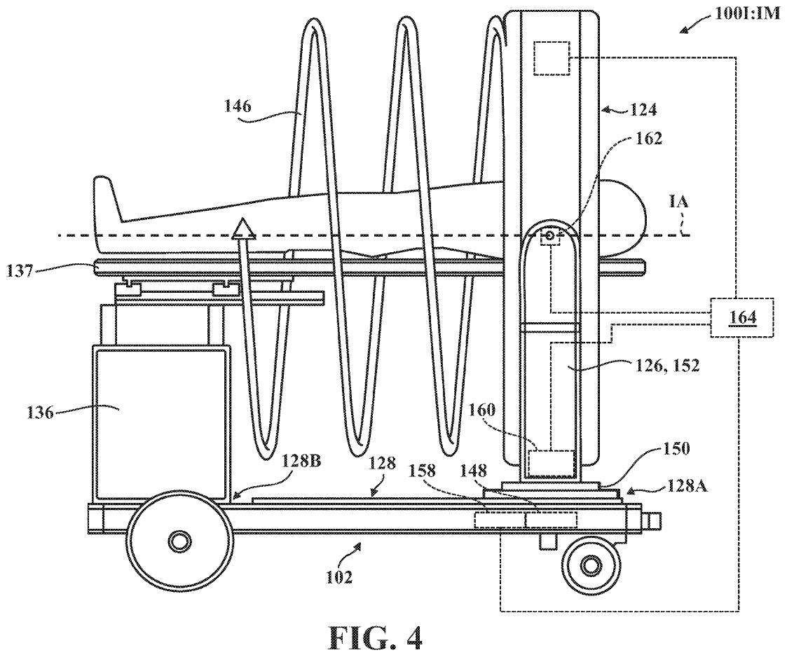

As shown in FIGS. 2A-2B, the mobile medical imaging system 100I may include a pedestal 136. The pedestal 136 may extend generally vertically upwards from the base 102 and be mounted to the base 102 adjacent to the second track end 128B to support a patient P above the base 102 (best shown in FIG. 4). For example, the pedestal 136 may be adapted to support a patient support 137 that can be attached to the pedestal 136. In one example, the patient support 137 is mounted to the pedestal 136 in a cantilevered manner and extends out into the imaging bore 134 of the imaging gantry 124 to support a patient P or other object being imaged. It will be understood that virtually any type of patient support 137 can be used in the present imaging system. For example, the present imaging system can utilize medical tables, and related accessories, of the type described in the JUPITER system brochure (11/2008) from TRUMPF Medezin Systeme GmbH & Co. KG of Puchheim, Germany, the entire contents of which are incorporated herein by reference. Furthermore, although the present examples illustrate patient supports 137 that can be used for medical imaging of human patients, it will be understood that the present invention encompasses any suitable tabletop support structure, including those designed for or suitable to support non-human subjects and non-living objects and materials. A plurality of different patient supports 137 can be attached and detached from the pedestal, where the tabletop supports are each customized for a particular application. Examples of different configurations of the pedestal 136 and patient support 137 are described in U.S. Patent Application Publication No. 2021/006,8775 entitled “Medical Imaging System and Methods,” the disclosure of which is hereby incorporated by reference in its entirety.

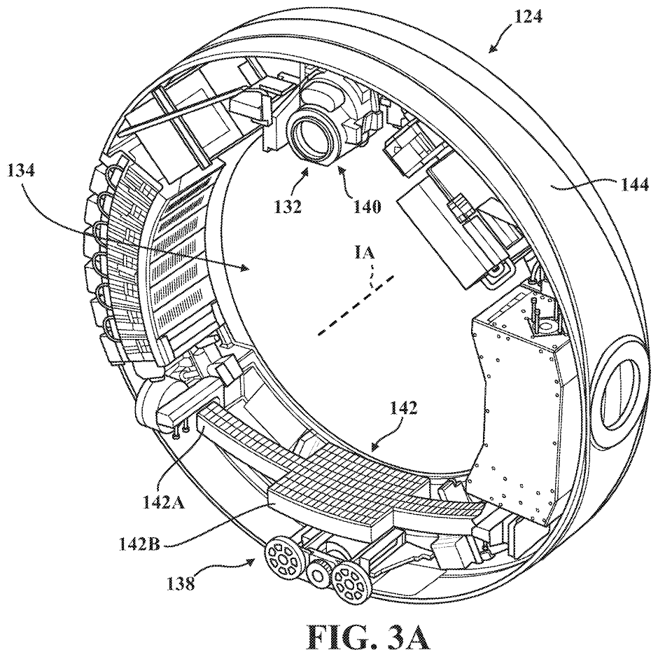

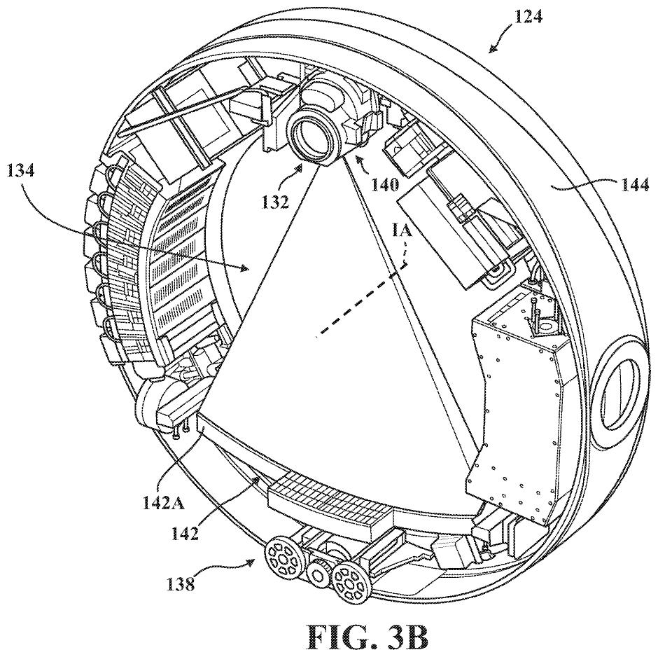

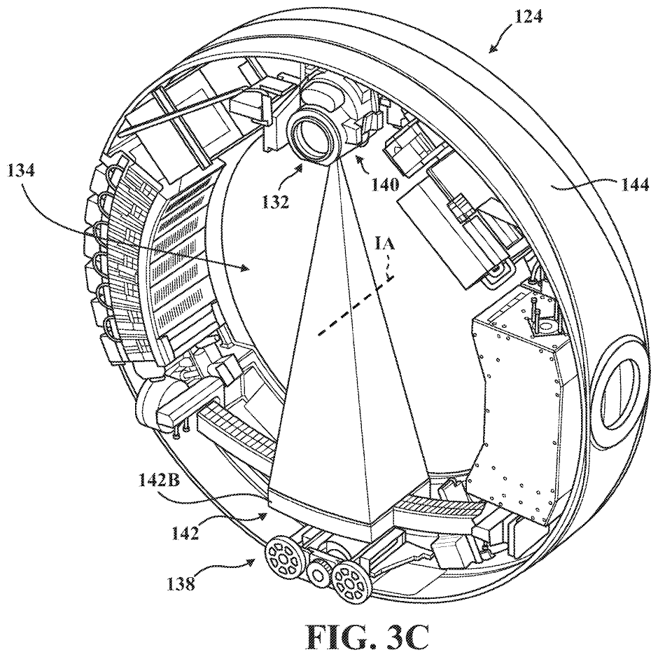

FIGS. 3A-3C show one example of the imaging gantry 124 including the at least one imaging component 132. In this example, the at least one imaging component 132 of the imaging gantry 124 includes a rotor 138 supporting an x-ray source 140 and a detector 142. The rotor 138 may be disposed within a gantry housing 144 defined by the imaging gantry 124 for rotation around the imaging bore 134. In some examples, the x-ray source 140 may be a fan-beam x-ray source (shown in FIG. 3B) and/or a cone-beam x-ray source (shown in FIG. 3C). The detector 142 may comprise an array of detectors 142. The array of detectors 142 may define an elongated first portion 142A for performing fan-beam CT imaging (e.g., axial and/or helical scans), a panel-shaped second portion 142B for performing 2D fluoroscopic imaging and/or 3D cone beam CT imaging, or a combination thereof, as shown in FIGS. 3A and 3B. FIG. 4 illustrates an example of the mobile medical imaging system 100I performing a helical scan. Here, the rotor 138 supporting the x-ray source 140 and the array of detectors 142 rotate around the imaging bore 134 of the gantry 124 to obtain imaging data, while the imaging gantry 124 and gantry mount 126 simultaneously translate along the base 102 from the first track end 128A to the second track end 128B (described in further detail below). The arrow 146 indicates the path of the at least one imaging component 132 around the imaging bore 134 in a helical scan. Notably, because the rotor 138 rotates around the imaging bore 134 during a scan, the motion of the rotor 138 may cause the mobile medical imaging system 100I to tilt or shift relative to the floor surface FS. Accordingly, the stabilization assembly 300 provides an additional point of contact with the floor surface FS to stabilize the mobile medical imaging system 100I.

Referring back to FIGS. 2A and 2B, as described above, the gantry mount 126 supports the imaging gantry 124 for movement along the track 128. The gantry mount 126 may be configured for movement between a plurality of track poses. The plurality of track poses may include a park pose PP, shown in FIG. 2A. In the park pose PP, the gantry mount is arranged adjacent to the first track end 128A. The mobile medical imaging system 100I also includes a translation mechanism 148 interposed between the base 102 and the imaging gantry 124 to drive the gantry mount 126 between the plurality of track poses in the imaging mode IM to acquire image data ID of a patient within the imaging bore 134, as shown in FIG. 4.

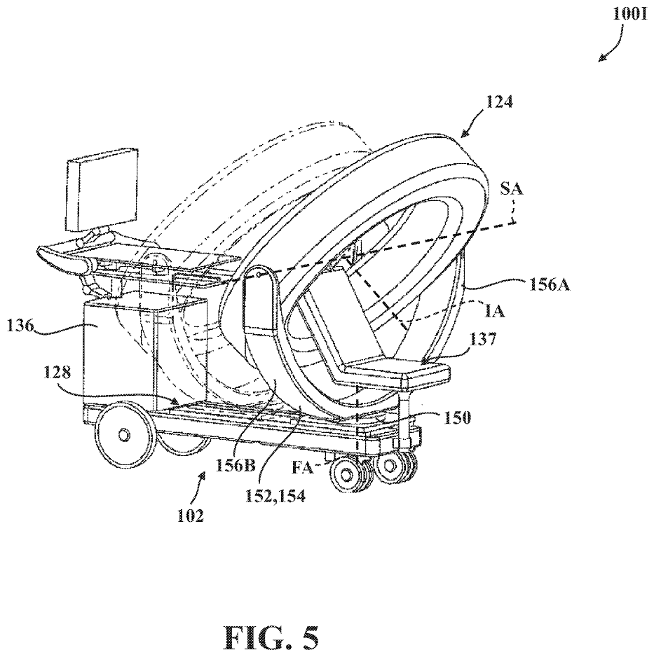

In some configurations, as best shown in FIGS. 5-7B, the gantry mount 126 includes a gantry mount base 150 that is operatively attached to the base 102, and a gantry mount member 152 operatively attached to the gantry mount base 150 for rotation relative to the gantry mount base 150 about a first axis FA. In these configurations, the gantry mount member 152 supports the imaging gantry 124 such that the gantry mount member 152 and the imaging gantry 124 are configured to rotate together about the first axis FA relative to the base 102. In some examples, the gantry mount member 152 includes a gimbal 154 having a pair of arms 156A, 156B (best shown in FIG. 5). Each of the arms 156A, 156B is coupled to an opposite side of the imaging gantry 124 to support the imaging gantry 124 above the base 102 and the gimbal 154. Additionally, referring to FIG. 5, in some configurations, the imaging gantry 124 may be configured to tilt about a second axis SA relative to the gimbal 154. Advantageously, allowing the imaging gantry 124 to tilt about the second axis SA may provide a number of different imaging configurations, such as shown in FIG. 5.

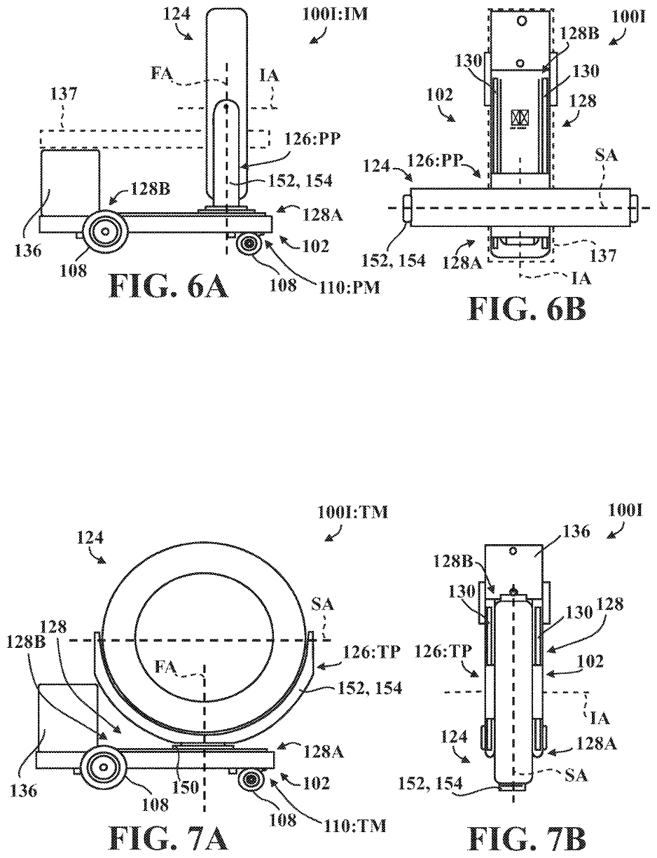

FIGS. 6A and 6B are side and top views, respectively, of the mobile medical imaging system 100I in the imaging mode IM and the gantry mount 126 in the park pose PP. While not shown in detail in FIGS. 6A and 6B, in the imaging mode IM, the base lift 110 may be in the parked mode PM such that the base 102 is stationary relative to the floor surface FS. In the park pose PP, the imaging axis IA is parallel to the track 128 and the gantry mount 126 is arranged adjacent to the first track end 128A. FIGS. 7A and 7B, show the gantry mount 126 of the mobile medical imaging system 100I in a transport pose TP. Although not shown in detail in FIGS. 7A and 7B, the base lift 110 may be in the transport mode TM to facilitate movement of the mobile medical imaging system 100I over the floor surface. In the transport pose TP, the gantry mount 126 is arranged between the first track end 128A and the second track end 128B and the gantry mount member 152 and the imaging gantry 124 are rotated about the first axis FA such that the imaging axis IA is transverse to the track 128. The profile of the mobile medical imaging system 100I is thus dramatically reduced in comparison to the imaging mode IM, such that the mobile medical imaging system 100I is typically only as wide as the width of the base 102 and/or the pedestal 136. This advantageously allows the mobile medical imaging system 100I to be more easily transported through narrow doors and hallways.

The mobile medical imaging system 100I can include one or more motors, as are known in the art, to control and effect the above-described motions. For example, as illustrated schematically in FIG. 4, the mobile medical imaging system 100I may include a translation motor 158 operatively attached to the translation mechanism 148 to drive the gantry mount 126 between the plurality of track poses, and a gantry motor 160 operatively attached to the gantry mount base 150 and the gantry mount member 152 for rotating the gantry mount member 152 relative to the base 102 about the first axis FA, and a gimbal motor 162 interposed between the gimbal 154 and the imaging gantry 124 for rotating the imaging gantry 124 relative to the gimbal 154 about the second axis SA. All of these respective motions can be controlled by a central computerized system controller 164. The system controller 164 may be included on the mobile medical imaging system 100I, such as housed inside the pedestal 136. In other examples, the system controller 164 may be located off the mobile medical imaging system 100I, such as in a mobile cart, and may comprise a general purpose computer programmed to provide the desired control functions and user interface, and is in electrical communication with the mobile medical imaging system 100I, such as via a cable or wireless link. The control system 164 can also control the operation of the at least one imaging component of the imaging gantry 124.

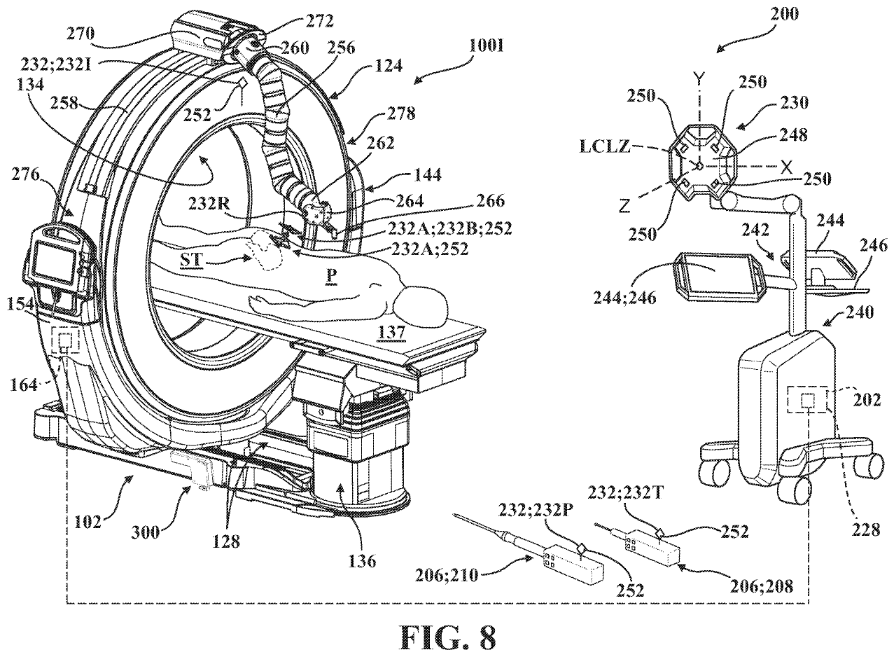

Referring to FIG. 8, the mobile medical imaging system 100I may further include a robotic system 200 for treating a patient P. The illustrated robotic system 200 generally includes a navigation system 202 one or more types of tools 206. As will be appreciated from the subsequent description below, the robotic system 200 is configured to, among other things, allow the surgeon to visualize, approach, and treat or otherwise manipulate anatomy of a patient P at a target site ST with a high level of control. To this end, imaging data ID of the target site ST may be acquired via the mobile medical imaging system 100I, and can be used to assist the surgeon in visualizing the patient's P anatomy at or otherwise adjacent to the target site ST. Here, the imaging data ID may also be utilized by the navigation system 202 to, among other things, facilitate navigation of tools 206 relative to the target site ST. Each of the components of the robotic system 200 introduced above will be described in greater detail below.

In FIG. 8, an operating room is shown with a patient P undergoing an exemplary surgical procedure performed using the robotic system 200. In this illustrative example, a minimally-invasive spinal surgical procedure, such as a posterior interbody spinal fusion, is being performed. It will be appreciated that this example is illustrative, and that other types of surgical procedures are contemplated. During the surgical procedure, one or more hand-held tools 206, such as a rotary tool 208 and/or a pointer tool 210, may be used by the surgeon. The tool 206 is for engaging the target site ST. As noted above and as is described in greater detail below, the navigation system 202 may be configured to track states of one or more of the tools 206 relative to the target site ST. In this exemplary surgical procedure, the rotary tool 208 may be employed as a cutting or drilling tool to remove tissue, form pilot holes (e.g., in the ilium, in vertebrae, and the like), or otherwise approach the target site ST. The rotary tool 208 may also be used to drive or otherwise install implantable components (e.g., pedicle screws, anchors, and the like).

For illustrative purposes, generically-depicted tools 206 configured for hand-held use are shown in FIG. 8. However, as will be appreciated from the subsequent description below, aspects of the robotic system 200 may be used with any suitable type of tool 206 without departing from the scope of the present disclosure. Furthermore, in addition to hand-held tools 206 of various types and configurations, aspects of the robotic system 200 may also be employed in connection with robotically-controlled tools 206 (not shown). Certain types of robotically-controlled tools 206 are disclosed in U.S. Pat. No. 9,119,655, entitled “Surgical Robotic arm Capable of Controlling a Surgical Instrument in Multiple Modes;” U.S. Pat. No. 10,456,207, entitled “Systems and Tools for use with Surgical Robotic Manipulators;” U.S. Pat. No. 11,160,620, entitled “End Effectors And Methods For Driving Tools Guided By Surgical Robotic Systems;” U.S. Pat. No. 10,959,783, entitled “Integrated Medical Imaging and Surgical Robotic System;” and U.S. Patent Application Publication No. 2020/0078097, entitled “Methods and Systems for Robot-Assisted Surgery,” the disclosures of each of which are hereby incorporated by reference in their entirety.

As noted above, the mobile medical imaging system 100I may be used to obtain imaging data ID of the patient, which may be a human or animal patient. In the representative version illustrated in FIG. 8, the mobile medical imaging system 100I is realized as an x-ray computed tomography (CT) imaging device configured to obtain raw x-ray imaging data ID of the patient P, as described above. The imaging data ID may be processed using the system controller 164, or another suitable controller, in order to construct three-dimensional imaging data ID, two-dimensional imaging data ID, and the like, which may be transmitted to or otherwise utilized by the navigation system 202 or other components of the robotic system 200.

In some versions, imaging data ID may be obtained preoperatively (e.g., prior to performing a surgical procedure) or intraoperatively (e.g., during a surgical procedure) by positioning the patient P within the imaging bore 134 of the mobile medical imaging system 100I. In order to obtain imaging data ID, a portion of the mobile medical imaging system 100I may be moved relative to the patient support 137 (described above) on which the patient P is disposed.

The robotic system 200 employs the navigation system 202 to, among other things, track movement of various objects, such as the tools 206 and parts of the patient's P anatomy (e.g., tissue at the surgical site ST), as well as portions of the mobile medical imaging system 100I in some versions. To this end, the navigation system 202 comprises a navigation controller 228 coupled to a localizer 230 that is configured to sense the position and/or orientation of trackers 232 within a localizer coordinate system LCLZ. In other words, the navigation system 202 includes the localizer 230 to track states of trackers 232 within a field of view. As is described in greater detail below, the trackers 232 (also referred to herein as “navigable trackers”) are fixed, secured, or otherwise attached to specific objects, and are configured to be monitored by the localizer 230.

The navigation controller 228 is disposed in communication with the localizer 230 and gathers position and/or orientation data for each tracker 232 sensed by the localizer 230 in the localizer coordinate system LCLZ. The navigation controller 228 may be disposed in communication with the system controller 164 e.g., to receive imaging data ID) and/or in communication with other components of the robotic system 200 (e.g., robotic arm controllers, tool controllers, and the like; not shown). However, other configurations are contemplated. The controllers 164, 228 may be realized as computers, processors, control units, and the like, and may be discrete components, may be integrated, and/or may otherwise share hardware.

It will be appreciated that the localizer 230 can sense the position and/or orientation of multiple trackers 232 to track correspondingly multiple objects within the localizer coordinate system LCLZ. By way of example, and as is depicted in FIG. 8, trackers 232 may comprise a tool tracker 232T, a pointer tracker 232P, an imaging system tracker 232I, one or more patient trackers 232A (e.g., a first patient tracker 232A, a second patient tracker 232B, and the like), a robot tracker 232R, as well as additional patient trackers, trackers for additional medical and/or surgical tools, and the like. The patient tracker 232A is adapted for attachment relative to the target site ST. One example of the robot tracker 232R is described in U.S. Provisional Patent Application 63/348,115 entitled “Robotic Surgical System with End Effector Marker Diffusers” which is incorporated by reference herein in its entirety.

The position of the patient trackers 232A, 232B relative to the anatomy of the patient P to which they are attached can be determined by known registration techniques, such as point-based registration in which the pointer tool 210 (to which the pointer tracker 232P is fixed) is used to touch off on bony landmarks on bone, or to touch off on several points across the bone for surface-based registration. Conventional registration techniques can be employed to correlate the pose of the patient trackers 232A, 232B to the patient's anatomy. Other types of registration are also possible.

Position and/or orientation data may be gathered, determined, or otherwise handled by the navigation controller 228 using conventional registration/navigation techniques to determine coordinates of trackers 232 within the localizer coordinate system LCLZ. These coordinates may be utilized by various components of the robotic system 200 (e.g., to facilitate control of the tools 206, to facilitate navigation based on imaging data ID, and the like).

In the representative version illustrated in FIG. 8, the navigation controller 228 and the localizer 230 are supported on a mobile cart 240 which is movable relative to the base 102 of the mobile medical imaging system 100I. Notably, as contemplated generally above although not shown, the mobile cart 240 may include a stabilization assembly 300 to prevent the mobile cart 240 from tilting or shifting relative to the floor surface FS, which may affect the accuracy of navigation. The mobile cart 240 also supports a user interface, generally indicated at 242, to facilitate operation of the navigation system 202 by displaying information to, and/or by receiving information from, the surgeon or another user. The user interface 242 may be disposed in communication with other components of the robotic system 200 (e.g., with the mobile medical imaging system 100I), and may comprise one or more output devices 244 (e.g., monitors, indicators, display screens, and the like) to present information to the surgeon (e.g., images, video, data, a graphics, navigable menus, and the like), and one or more input devices 246 (e.g., buttons, touch screens, keyboards, mice, gesture or voice-based input devices, and the like).

In some versions, the robotic system 200 is capable of displaying a virtual representation of the relative positions and orientations of tracked objects to the surgeon or other users of the robotic system 200, such as with images and/or graphical representations of the anatomy of the patient P and the tool 206 presented on one or more output devices 244 (e.g., a display screen). The navigation controller 228 may also utilize the user interface 242 to display instructions or request information from the surgeon or other users of the robotic system 200. Other configurations are contemplated. One type of mobile cart 240 and user interface 242 of this type of navigation system 202 is described in U.S. Pat. No. 7,725,162, entitled “Surgery System,” the disclosure of which is hereby incorporated by reference in its entirety.

Because the mobile cart 240 and the imaging gantry 124 of the mobile medical imaging system 100I can be positioned relative to each other and also relative to the patient P in the representative version illustrated in FIG. 8, the navigation system 202 can transform the coordinates of each tracker 232 from the localizer coordinate system LCLZ into other coordinate systems (e.g., defined by different trackers 232, localizers 230, and the like), or vice versa, so that navigation relative to the target site ST (or control of tools 206) can be based at least partially on the relative positions and orientations of multiple trackers 232 within a common coordinate system (e.g., the localizer coordinate system LCLZ). Coordinates can be transformed using a number of different conventional coordinate system transformation techniques. It will be appreciated that the localizer 230 or other components of the navigation system 202 could be arranged, supported, or otherwise configured in other ways without departing from the scope of the present disclosure. By way of non-limiting example, the localizer 230 could be coupled to the mobile medical imaging system 100I in some versions (e.g., to the imaging gantry 124). Other configurations are contemplated.

In the illustrated version, the localizer 230 is an optical localizer and includes a camera unit 248 with one or more optical position sensors 250. The navigation system 202 employs the optical position sensors 250 of the camera unit 248 to sense the position and/or orientation of the trackers 232 within the localizer coordinate system LCLZ. To this end, the trackers 232 each employ one or more markers 252 (also referred to as “fiducials” in some versions) that are supported on an array in a predetermined arrangement. However, as will be appreciated from the subsequent description below, trackers 232 may have different configurations, such as with different quantities of markers 252 that can be secured to or otherwise formed in other structures besides arrays (e.g., various types of housings, frames, surfaces, and the like). Other configurations are contemplated.

In some versions, certain trackers 232 (e.g., the patient tracker 232A) may employ “passive” markers 252 (e.g., reflective markers such as spheres, cones, and the like) which reflect emitted light that is sensed by the optical position sensors 250 of the camera unit 248. In some versions, trackers 232 employ “active” markers 252 (e.g., light emitting diodes “LEDs”), which emit light that is sensed by the optical position sensors 250 of the camera unit 248. Examples of navigation systems 202 of these types are described in U.S. Pat. No. 9,008,757, entitled “Navigation System Including Optical and Non-Optical Sensors,” the disclosure of which is hereby incorporated by reference in its entirety.

Although one version of the mobile cart 240 and localizer 230 of the navigation system 202 is illustrated in FIG. 8, it will be appreciated that the navigation system 202 may have any other suitable configuration for monitoring trackers 232 which may be of various types and configurations and could employ various types of markers 252. Thus, for the purposes of clarity and consistency, the term “marker 252” is used herein to refer to a portion of a tracker 232 (e.g., a passive or active marker 252 mounted to an array or otherwise coupled to a tracked object) that can be monitored by a localizer 230 to track (e.g., states, motion, position, orientation, and the like) of the object to which the tracker 232 is secured, irrespective of the specific type or configuration of the localizer 230 and/or tracker 232.

In some versions, the navigation system 202 and/or the localizer 230 could be radio frequency (RF) based. For example, the navigation system 202 may comprise an RF transceiver coupled to the navigation controller 228. Here, certain trackers 232 may comprise markers 252 realized as RF emitters or transponders, which may be passive or may be actively energized. The RF transceiver transmits an RF tracking signal, and the RF emitters respond with RF signals such that tracked states are communicated to (or interpreted by) the navigation controller 228. The RF signals may be of any suitable frequency. The RF transceiver may be positioned at any suitable location to track the objects using RF signals effectively. Furthermore, it will be appreciated that versions of RF-based navigation systems may have structural configurations that are different than the navigation system 202 illustrated throughout the drawings.

In some versions, the navigation system 202 and/or localizer 230 may be electromagnetically (EM) based. For example, the navigation system 202 may comprise an EM transceiver coupled to the navigation controller 228. Here, certain trackers 232 may comprise markers 252 realized as EM components (e.g., various types of magnetic trackers, electromagnetic trackers, inductive trackers, and the like), which may be passive or may be actively energized. The EM transceiver generates an EM field, and the EM components respond with EM signals such that tracked states are communicated to (or interpreted by) the navigation controller 228. The navigation controller 228 may analyze the received EM signals to associate relative states thereto. Here too, it will be appreciated that versions of EM-based navigation systems may have structural configurations that are different than the navigation system 202 illustrated throughout the drawings.

Those having ordinary skill in the art will appreciate that the navigation system 202 and/or localizer 230 may have any other suitable components or structure not specifically recited herein. Furthermore, any of the techniques, methods, and/or components described above with respect to the camera-based navigation system 202 shown throughout the drawings may be implemented or provided for any of the other versions of the navigation system 202 described herein. For example, the navigation system 202 may also be based on one or more of inertial tracking, ultrasonic tracking, image-based optical tracking (e.g., with markers 252 are defined by patterns, shapes, edges, and the like that can be monitored with a camera), or any combination of tracking techniques. Other configurations are contemplated.

With continued reference to FIG. 8, the robotic system 200 may include a robotic arm 256 operatively attached to a support element 258 and configured to maintain alignment of the tool 206 relative to the target site ST. The robotic arm 256 may extend between a base end 260 and a mount end 262 arranged for movement relative to the base end 260. The robotic system 200 may further includes an end effector 264 attached to the mount end 262 of the robotic arm 256 and configured to support one or more types of tools 206, instruments, and the like. More specifically, the robotic system 200 may further include a tool guide 266 supported by the end effector 264, and the tool guide 266 may be configured to support the tool 206 relative to a trajectory that is aligned or otherwise determined relative to the surgical site ST on the patient P.

The robotic arm 256 may comprise a multi-joint arm that includes a plurality of linkages connected by joints having actuator(s) and optional encoder(s) (not shown in detail) to enable the linkages to bend, rotate and/or translate relative to one another in response to control signals from a robot control system. The robotic arm 256 may be fixed to the mobile medical imaging system 100I, such as on the support element 258 (e.g. a curved rail) that may extend concentrically over the outer surface of the imaging gantry 124 of the mobile medical imaging system 100I and that may be located close to the target site ST of the patient P. In some versions, the robotic arm 256 could be coupled to a mobile cart (not shown) or to another type of support element 258 that is not necessarily coupled to the mobile medical imaging system 100I. As broadly contemplated above although not shown, in these example, the robotic arm 256 may be coupled to a separate base 102 including the stabilization assembly 300 for ensuring that the robotic arm 256 does not shift or tilt relative to the floor surface FS during operation of the robotic arm 256. Although a single robotic arm 256 is shown in FIG. 8, it will be understood that the robotic system 200 may include multiple robotic arms attached to suitable support structure(s). Other configurations are contemplated.

The support element 258 may form a semicircular arc and may be concentric with the outer circumference of the imaging gantry 124. The support element 258 may extend around at least 25%, such as between about 30-50% of the outer circumference of the imaging gantry 124. The support element 258 may extend around at least a portion of the outer circumference of the imaging gantry 124 that is located above the target site ST of the patient P. More specifically, the base end 260 of the robotic arm 256 (e.g., the end of the robotic arm 256 opposite the end effector 264) may be fixed to the support element 258, in a non-limiting example, at a position that is less than about 2 meters, such as less than about 1 meter (e.g., between 0.5 and 1 meter) from the surgical site ST of the patient P during a surgical procedure.

In versions, the support element 258 may extend along a semicircular arc having a radius that is greater than about 33 inches, such as greater than about 35 inches (e.g., between 33 and 50 inches). The support element 258 may be spaced from the outer surface of the imaging gantry 124 by a pre-determined distance, which may be from less than an inch (e.g., 0.5 inches) to 6 or 10 inches or more. In some versions, the support element 258 may be spaced from the imaging gantry 124 by an amount sufficient to enable the tilt motion of the imaging gantry 124 with respect to the gimbal 154 supporting the imaging gantry 124 over at least a limited range of motion. Additionally, in some versions, the support element 258 may comprise one or more straight segments (e.g., rail segments), where at least a portion of the support element 258 may extend over the top surface of the imaging gantry 124. Other configurations are contemplated.

A carriage 270 may be located on the support element 258 and may include a mounting surface 272 for mounting the base end 260 of the robotic arm 256 to the carriage 270. As shown in FIG. 8, the carriage 270 may extend from the support element 258 towards a first (e.g., front) face of the imaging gantry 124. The mounting surface 272 for the robotic arm 256 may extend beyond the first (e.g., front) face of the imaging gantry 124 and the robotic arm 256 may extend over the first (e.g., front) face of the imaging gantry 124. In some versions, the configuration of the carriage 270 and the mounting surface 272 may be reversed such that the mounting surface 272 extends beyond the second (e.g., rear) face of the imaging gantry 124, and the robotic arm 256 may extend over the second (e.g., rear) face of the imaging gantry 124. In this configuration, the patient support 137 may be configured such that the patient support 137 and patient P extend into or through the imaging bore 134, and a portion of the patient P requiring surgical intervention (e.g., the cranium) may be accessed from the second (e.g., rear) side of the imaging gantry 124.

In some versions, the carriage 270 and the robotic arm 256 attached thereto may be moved to different positions along the length of support element 258 (e.g., any arbitrary position between a first end 276 and a second end 278 of the support element 258). The carriage 270 and the robotic arm 256 may be fixed in place at a particular desired position along the length of the support element 258. In some versions, the carriage 270 may be moved manually (e.g., positioned by an operator at a particular location along the length of the support element 258 and then clamped or otherwise fastened in place). Alternately, the carriage 270 may be driven to different positions using a suitable drive mechanism (e.g., a motorized belt drive, friction wheel, gear tooth assembly, cable-pulley system, etc., not shown in detail). The drive mechanism may be located on the carriage 270 and/or the support element 258, for example. An encoder mechanism may be utilized to indicate the position of the carriage 270 and the base end 260 of the robotic arm 256 on the support element 258. Although the version of FIG. 8 illustrates one robotic arm 256 mounted to the support element 258, it will be understood that more than one robotic arm 256 may be mounted to the support element 258 via respective carriages 270.

In some versions, the robotic arm 256 may be mounted directly to the support element 258, such as on a mounting surface 272 that is integrally formed on the support element 258. In such an version, the position of robotic arm 256 may not be movable along the length of the support element 258. In other versions, the robotic arm 256 may be secured to any other portion of the mobile medical imaging system 100I, such as directly mounted to the imaging gantry 124. Alternatively, the robotic arm 256 may be mounted to the patient support 137 or pedestal 136, to any of the wall, ceiling or floor in the operating room, or to a separate cart as noted above. In some versions, the robotic arm 256 may be mounted to a separate mobile shuttle, similar to as is described in U.S. Pat. No. 11,103,990, entitled “System and Method for Mounting a Robotic Arm in a Surgical Robotic System,” the disclosure of which is hereby incorporated by reference in its entirety. Although a single robotic arm 256 is shown in FIG. 8, it will be understood that two or more robotic arms 256 may be utilized.

Those having ordinary skill in the art will appreciate that the robotic arm 256 can be employed to aid in the performance of various types of surgical procedures, such as a minimally-invasive spinal surgical procedure or various other types of orthopedic, neurological, cardiothoracic and general surgical procedures. In the version of FIG. 8, the robotic arm 256 may be used to assist a surgeon performing a surgical procedure in the lumbar spinal region of a patient. The robotic arm 256 may also be used for thoracic and/or cervical spinal procedures. The procedures may be performed posteriorly, anteriorly or laterally. Other configurations are contemplated.

In some versions, the robotic arm 256 may be controlled to move the end effector 264 to one or more pre-determined positions and/or orientations with respect to a patient P, such as to and/or along a trajectory defined relative to the anatomy of the patient P. As discussed above, the end effector 264 may be realized as or may otherwise support various types of instruments and/or tools 206 including, but not limited to, a needle, a cannula, a dilator, a cutting or gripping instrument, a scalpel, a drill, a screw, a screwdriver, an electrode, an endoscope, an implant, a radiation source, a drug, etc., that may be inserted into the body of the patient P. In some versions, the end effector 264 may be realized as a hollow tube or cannula configured to receive a surgical tool 206, including without limitation a needle, a cannula, a dilator, a cutting or gripping instrument, a scalpel, a drill, a screw, a screwdriver, an electrode, an endoscope, an implant, a radiation source, a drug, and the like. The surgical tool 206 may be inserted into or otherwise adjacent to the patient's body through the hollow tube or cannula by a surgeon. The robotic arm 256 may be controlled to maintain the position and orientation of the end effector 264 with respect to the patient P to ensure that the surgical tool(s) 206 follow a desired trajectory through the patient's body to reach the target site ST. The target site ST may be determined preoperatively and/or intraoperatively, such as during a surgical planning process, based on patient images which may be obtained using the mobile medical imaging system 100I.

In the representative version illustrated herein, the navigation system 202 tracks the robotic arm 256 within the localizer coordinate system LCLZ via the robot tracker 232R. To this end, a control loop may continuously read the tracking data and current parameters (e.g., joint parameters) of the robotic arm 256, and may send instructions to the navigation controller 228 and/or to the system controller 164 (and/or some other controller, such as a robot controller) to cause the robotic arm 256 to move to a desired position and orientation within the localizer coordinate system LCLZ.

In some versions, a surgeon may use one or more portions of the robotic system 200 as a planning tool for a surgical procedure, such as by setting trajectories within the patient for inserting tools 206, as well as by selecting one or more target sites ST for a surgical intervention within the patient's body. The trajectories and/or target sites ST set by the surgeon may be saved (e.g., in a memory of a computer device) for later use during surgery. In some versions, the surgeon may be able to select stored trajectories and/or target sites ST using the robotic system 200, and the robotic arm 256 may be controlled to perform a particular movement based on the selected trajectory and/or target site ST. For example, the robotic arm 256 may be moved to position the end effector 264 of the robotic arm 256 into alignment with the pre-defined trajectory and/or over the pre-determined target site ST. As discussed above, the end effector 264 may include the tool guide 266 which may be used to guide the tool 206 relative to the patient's body along the pre-defined trajectory and/or to the pre-defined target site ST.

As discussed above, the localizer 230 may include a camera unit 248 with one or more optical position sensors 250. More specifically, the optical position sensors 250 may be light sensors capable of sensing changes in infrared (IR) emitted within a field of view. In some versions, the localizer 230 may include one or more radiation sources (e.g., one or more diode rings) that direct radiation (e.g., IR radiation) into the surgical field, where the radiation may be reflected by the markers 252 and received by the cameras. In the illustrated version, certain active markers 252 (e.g., active markers 252 which define the robot tracker 232R) are configured to emit IR light detectable by the optical position sensors 250 of the localizer 230. The navigation controller 228 may be coupled to the localizer 230 and may determine the positions and/or orientations of markers 252 detected by the optical position sensors 250 using, for example, triangulation and/or transformation techniques. A 3D model and/or mathematical simulation of the surgical space may be generated and continually updated using motion tracking software implemented by the navigation controller 228.

Additionally, the patient tracker 232A may be rigidly attached to a portion of the patient's anatomy in the anatomical region of interest adjacent to the target site ST (e.g., clamped or otherwise attached to the ilium, to the spinous process of the vertebrae, and the like) to enable the anatomical region of interest to be continually tracked by the navigation system 202. In the illustrated version, the robot tracker 232R is rigidly attached to the end effector 264 of the robotic arm 256 to enable the robotic arm 256 to be tracked using the navigation system 202. Using the pose of the end effector tracker 282 (as well as of the patient tracker 232) monitored within the localizer coordinate system LCLZ by the localizer 230, the navigation controller 228 and/or some other controller (e.g., a robot controller) may include software configured to perform transformations between joint coordinates of the robotic arm 256 and the localizer coordinate system LCLZ which, in turn, may be utilized by the robotic arm 256 to control or otherwise adjust the position and/or orientation of the end effector 264 with respect to the patient P. In some versions, the robotic arm 256 may include multiple robot trackers 232R and/or robot trackers 232R other than the end effector tracker 282 (e.g., on joints of the arm). Other configurations are contemplated.

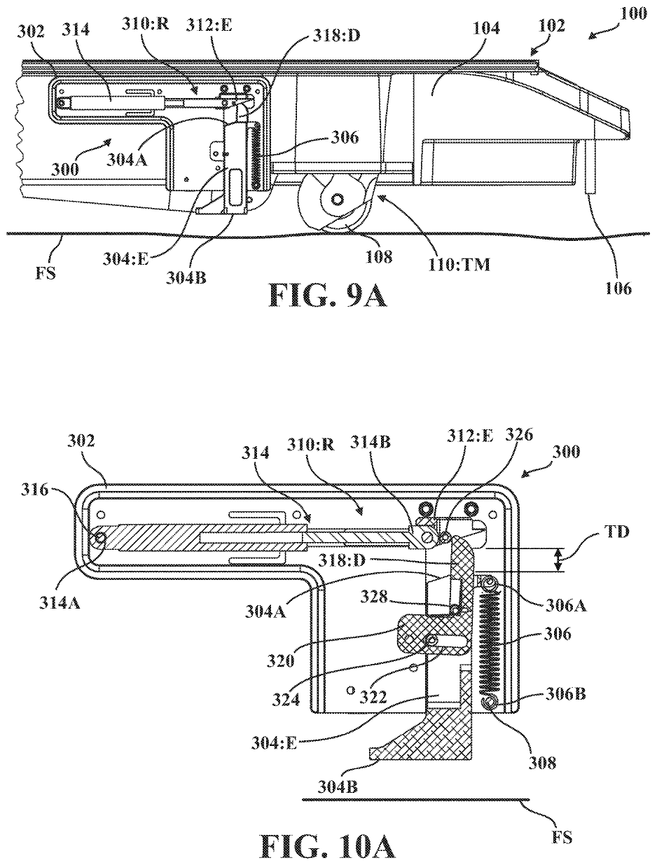

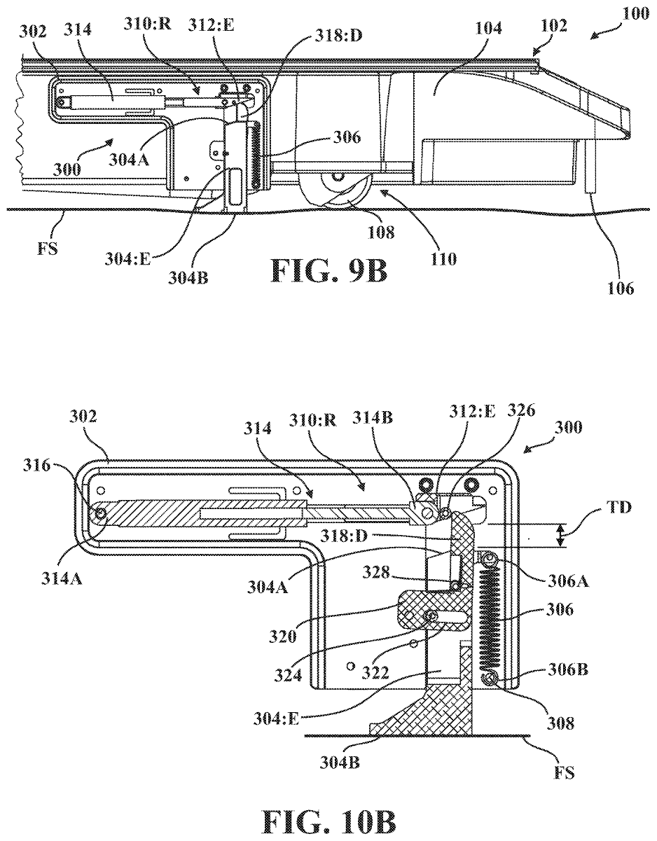

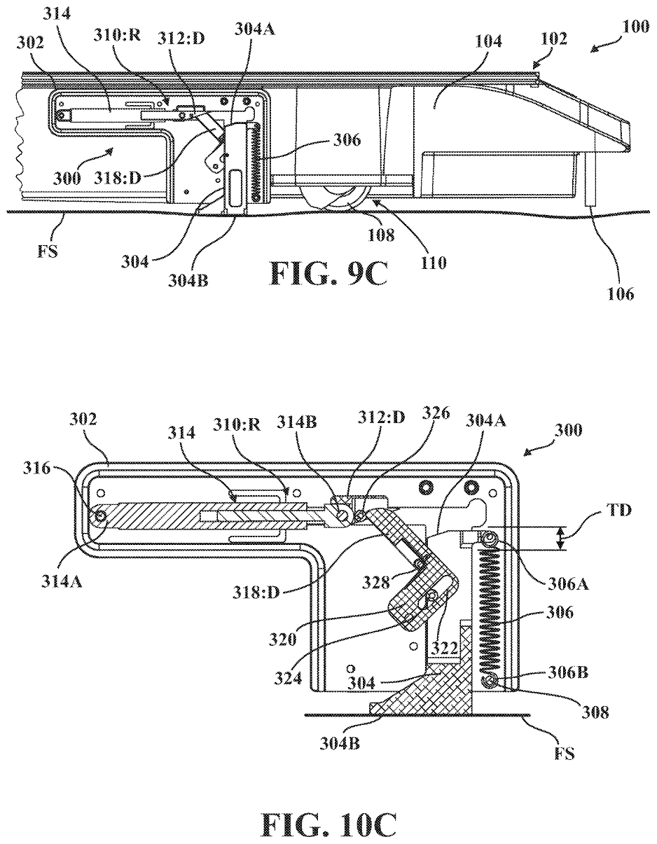

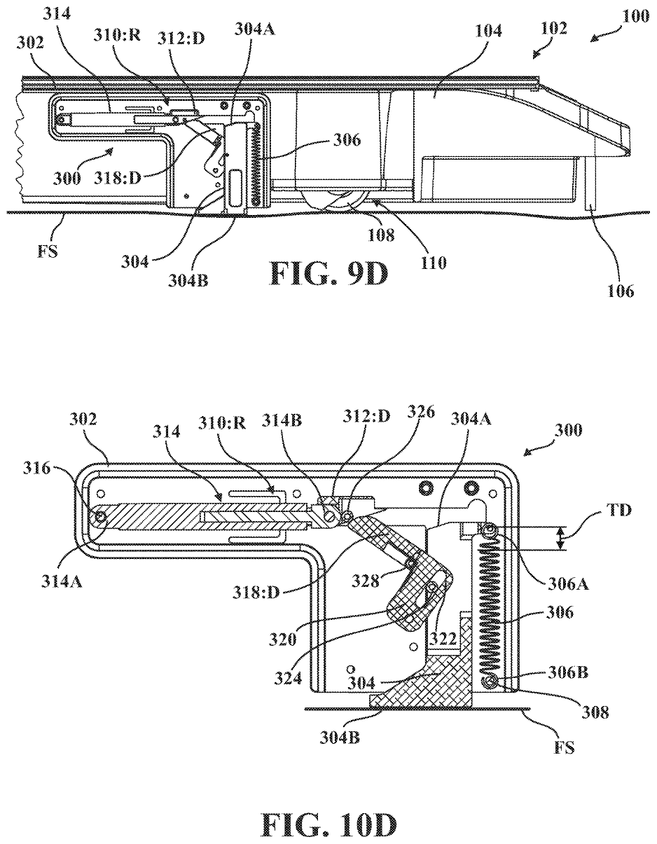

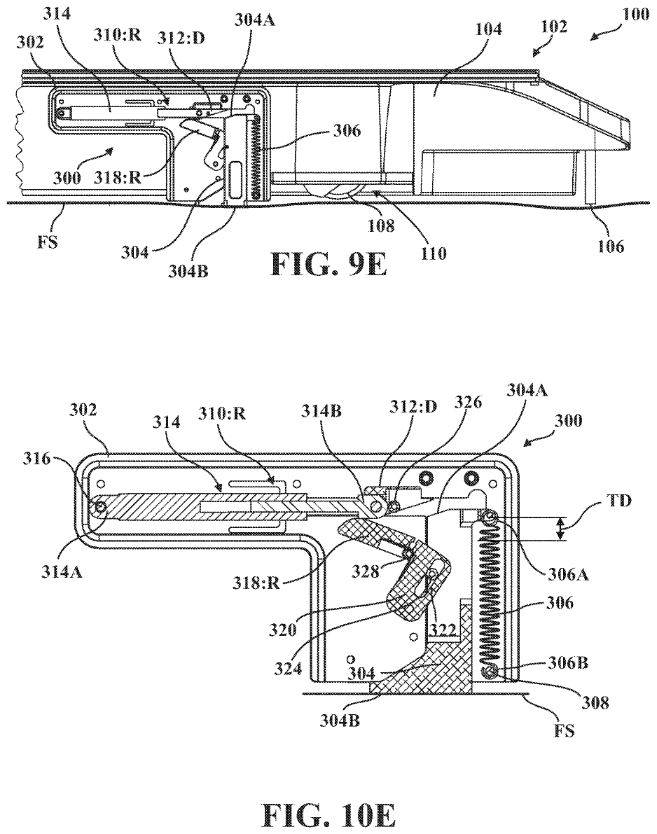

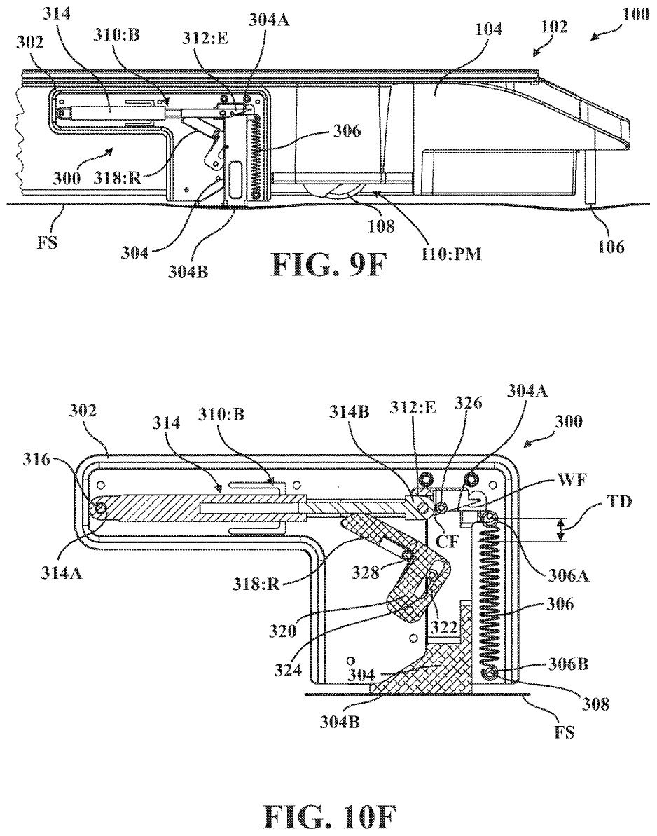

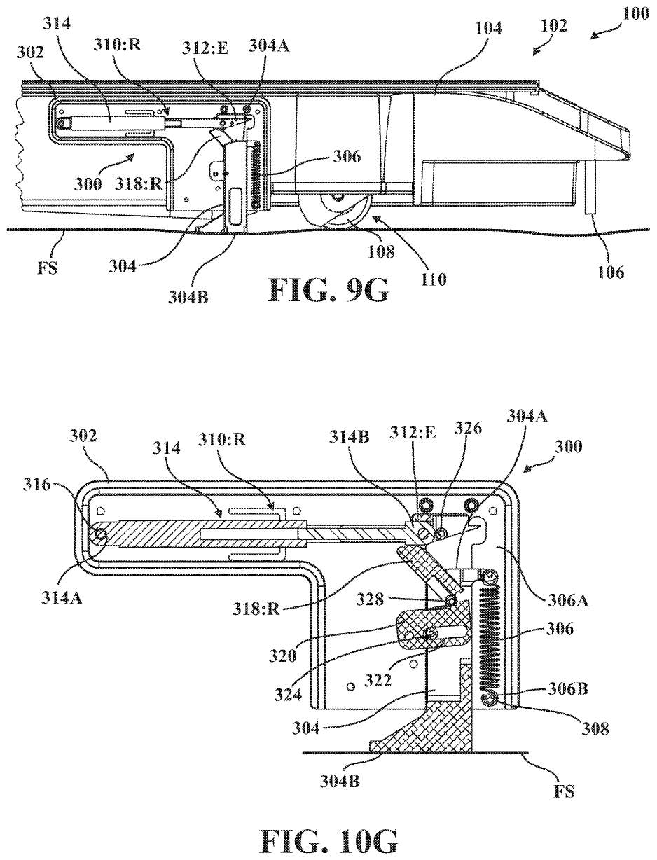

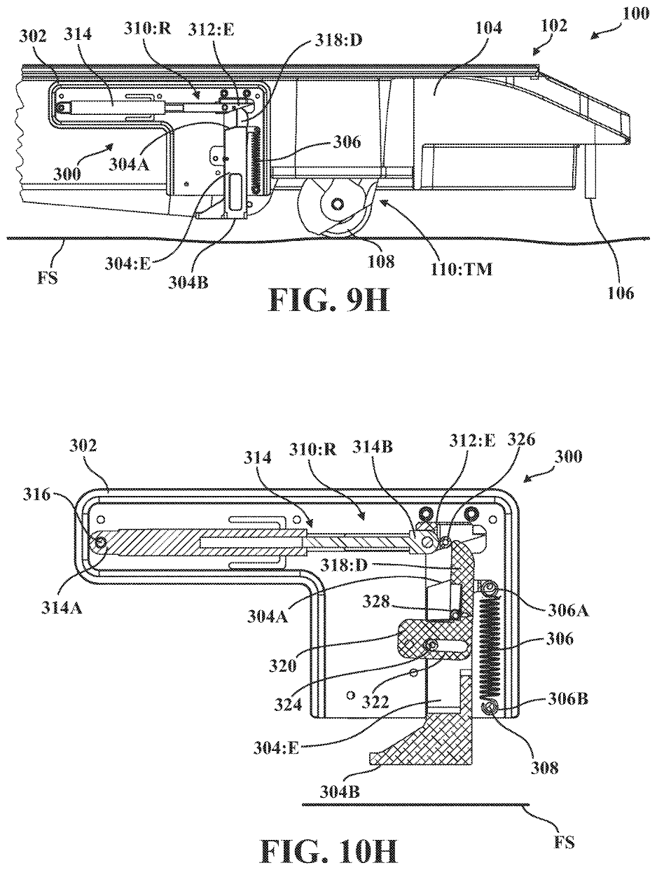

Referring to FIGS. 1A and 1B and FIGS. 9A through 10H, the stabilization assembly 300 includes a stabilization housing 302 coupled to the base 102, and a foot 304 extending between a top end 304A and a bottom end 304B and supported for displacement relative to the stabilization housing 302 between a plurality of foot positions. The plurality of foot positions includes an extended foot position 304E. As best shown in FIGS. 1A and 9A, when the base lift 110 is in the transport mode TM and the foot 304 is in the extended foot position 304E, the bottom end 304B of the foot 304 is arranged vertically between the contact surface 106 of the base 102 and the floor surface FS. FIG. 1B shows the base lift 110 of the base 102 in the parked mode PM and the foot 304 of the stabilization assembly 300 contacting the floor surface FS to provide an additional point of contact to ensure that the mobile medical system 100 does not shift or tilt relative to the floor surface FS during operation of the mobile medical system 100, as described in further detail below in the context of FIGS. 9A through 10H.

The stabilization assembly 300 also includes a foot biasing element 306 that is operatively attached to the foot 304 to urge the foot 304 towards the extended foot position 304E. For example, referring to FIG. 10A, the foot biasing element 306 may extend between a first foot biasing element end 306A and a second foot biasing element end 306B. The first foot biasing element end 306A may be operatively attached to the foot 304 to urge the foot 304 towards the extended foot position 304E, and the second biasing element end 306B may be coupled to a foot biasing mount 308 defined by the stabilization housing 302. Other configurations of arranging the foot biasing element 306 are contemplated. Additionally, while the foot biasing element 306 is schematically illustrated as a spring in FIGS. 9A through 10H, other suitable configurations for the foot biasing element 306 to urge the foot 304 towards the extended foot position 304E are contemplated.

The stabilization assembly 300 further includes a retainer 310. As described in further detail below, the retainer 310 is operable between a released state 310R (shown in FIGS. 9A through 10E and FIGS. 9G through 10H) and a brace state 310B (shown in FIGS. 9F and 10F). In the released state 310R, the retainer 310 permits movement of the foot 304 relative to the stabilization housing 302. In the brace state 310B, the retainer 310 inhibits movement of the foot 304 away from the floor surface FS. As described in further detail below, the retainer 310 is configured to change operation from the released state 310R to the brace state 310B in response to movement of the foot 304 beyond a threshold displacement TD from the extended foot position 304E occurring in response to abutment of the foot 304 with the floor surface FS as the base lift 110 moves from the transport mode TM towards the parked mode PM.

Referring to FIGS. 9A through 10H, in some configurations, the retainer 310 may further include a chock 312 arranged for movement between an engaged position 312E and a disengaged position 312D. As best shown in FIGS. 9F and 10F, when the retainer 310 is in the brace state 310B and the chock 312 is in the engaged position 312E, the chock 312 abuts the top end 304A of the foot 304 to inhibit movement of the foot 304 away from the floor surface FS. In the disengaged position 312D, the chock 312 is spaced from the top end 304A of the foot 304 to permit movement of the foot 304 relative to the stabilization housing 302 when the retainer 310 is in the released state 310R. The chock 312 may define any suitable shape to abut the top end 304A of the foot 304 to 304 to inhibit movement of the foot 304 away from the floor surface FS when the retainer 310 is in the brace state 310B.

Still referring to FIGS. 9A through 10H, in some configurations, the retainer 310 may further include a retainer biasing element 314. The retainer biasing element 314 may be disposed in the stabilization housing 302 and operatively attached to the chock 312 to urge the chock 312 to the engaged position 312E. For example, as shown in FIGS. 9A through 10H, in one configuration, the retainer biasing element 314 extends between a first end 314A that is coupled to a retainer biasing mount 316 that is defined by the stabilization housing 302, and a second end 314B that is operatively attached to the chock 312. While the retainer biasing element 314 is illustrated as a gas spring in FIGS. 9A through 10H, other suitable configurations to urge the chock 312 towards the engaged foot position 304E are contemplated. Additionally, in some examples, the retainer 310 may further comprise a damper configured to slow translation of the chock 312 from the disengaged position 312D to the engaged position 312E (described in further detail below in the context of FIGS. 9D through 10F). In the examples illustrated in FIGS. 9A through 10H, the damper is integral with the retainer biasing element 314. However, it should be appreciated that in other configurations, the damper may be a separate component from the retainer biasing element 314.

FIGS. 9A through 10H show the stabilization assembly 300 with part of the stabilization housing 302 hidden to reveal the internal componentry of the stabilization assembly 300. Additionally, FIGS. 9A through 10H illustrate a sequence of the operation of the stabilization assembly 300 as the base lift 110 of the mobile medical system 100 moves between the transport mode TM and the parked mode PM. The sequence from FIGS. 9A and 10A to 9B and 10B shows the stabilization assembly 300 beginning to contact the floor surface FS to provide the mobile medical system 100 with an additional point of contact with the floor surface FS. FIGS. 9A and 10A show the base lift 110 in the transport mode TM. Accordingly, as described above, the foot biasing element 306 urges the foot 304 to the extended foot position 304E such that the bottom end 304B of the foot 304 is arranged vertically between the contact surface 106 and the floor surface FS (i.e., the bottom end 304B of the foot is spaced from the floor surface FS and arranged vertically below the contact surface 106). FIGS. 9B and 10B show the base lift 110 moving from the transport mode TM toward the parked mode PM. In other words, FIGS. 9B and 10B show the base lift 110 lowering the base 102 relative to the floor surface FS such that the stabilization assembly 300 and the contact surface 106 move toward the floor surface FS. As a result, FIGS. 9B and 10B show the bottom end 304B of the foot 304 making initial contact with the floor surface FS. The sequence from FIGS. 9B and 10B to 9C and 10C show the foot 304 displacing away from the extended foot position 304E in response to abutment of the foot 304 with the floor surface FS as the base lift 110 continues to move toward the parked mode PM. In other words, as the base lift 110 continues to lower the base 102 relative to the floor surface FS, the foot 304 displaces from the extended foot position 304E as shown in FIGS. 9B and 10B such that the foot 304 retracts into the stabilization housing 302.

In some examples, the retainer 310 further includes a finger 318 that is operatively attached to the foot 304. Referring to FIGS. 9A through 10B, the finger 318 may be configured to engage the chock 312 when the foot 304 is in the extended foot position 304E. The finger 318 may be operatively attached to the foot 304 for coordinated movement with the foot 304. For example, the finger 318 may be operatively attached to the foot 304 via a finger lever 320. The finger lever 320 may be pivotably attached to the stabilization housing 302 and support the finger 318. The finger lever 320 may also define a slot 322, and the foot 304 may define a post 324 that is disposed in the slot 322 such that the finger lever 320 moves in a coordinated manner with the foot 304. For example, referring to the sequence from FIGS. 9B and 10B to 9C and 10C, the post 324 moves within the slot 322 as the foot 304 displaces from the extended foot position 304E such that the finger lever 320 pivots relative to the stabilization housing 302 to move the finger 318. As a result, referring to FIGS. 9C and 10C, the finger 318 may be configured to displace the chock 312 toward the disengaged position 312D as the foot 304 displaces from the extended foot position 304E in response to abutment of the foot 304 with the floor surface FS as the base lift 110 continues to move toward the parked mode PM. Other configurations of displacing the chock 312 toward the disengaged position 312D as the foot 304 displaces from the extended foot position 304E in response to abutment of the foot 304 with the floor surface FS are contemplated.

Next, the sequence from FIGS. 9C and 10C to 9D and 10D show the foot 304 continuing to displace away from the extended foot position 304E in response to abutment of the foot 304 with the floor surface FS as the base lift 110 continues to move toward the parked mode PM. Referring to FIGS. 9D and 10D, the finger 318 is configured to disengage from the chock 312 as the foot 304 reaches the threshold displacement TD from the extended foot position 304E. For example, in the sequence from Figured 9C and 10C to 9D and 10D, the finger 318 may be configured to slip off of a roller 326 that is supported by the chock 312. Additionally, in some examples, the finger 318 may be attached to the finger lever 320 for pivoting motion relative to the finger lever 320 between a deployed position 318D and a retracted position 318R. FIGS. 9A through 10B show the finger in the deployed position 318D. In the deployed position 318D, the finger 318 is arranged to engage the chock 312 when the foot 304 is in the extended foot position 304E to displace the chock 312 toward the disengaged position 312D as the foot 304 displaces from the extended foot position 304E in response to abutment of the foot 304 with the floor surface FS. The sequence from FIGS. 9D and 10D to 9E and 10E show the finger pivoting to the retracted position 318R. In the retracted position 318R, the finger 318 pivots relative to the finger lever 320 in response to the finger disengaging from the chock 312 to allow the chock 312 to translate toward the engaged position 312E to inhibit movement of the foot 304 away from the floor surface FS (i.e., to place the retainer 310 in the brace state 310B). Other configurations for disengaging the finger 318 from the chock 312 as the foot 304 reaches the threshold displacement TD from the extended foot position 304E are contemplated.

The sequence from FIGS. 9E and 10E to 9F and 10F show the base lift 110 reaching the parked mode PM such that the contact surface 106 is at least partially supporting the mobile medical system 100 on the floor surface FS. As a result of the finger 318 disengaging from the chock 312 (as described above) the retainer biasing element 314 urges the chock 312 to the engaged position 312E (shown in FIGS. 9F and 10F) to bring the chock 312 into abutment with the top end 304A of the foot 304 to inhibit movement of the foot 304 away from the floor surface FS. Here, notably, the damper (described above) may slow translation of the chock 312 toward the engaged position 312E to allow time for the foot 304 to reach its final resting place in abutment with the floor surface FS at the base lift 110 reaches the parked mode PM. Once the chock 312 reaches the engaged position 312E, the retainer 310 is in the brace state 310B to inhibit movement of the foot 304 away from the floor surface FS. Accordingly, FIGS. 9F and 10F illustrate the retainer 310 in the brace state 310B to inhibit movement of the foot 304 away from the floor surface FS. As a result, the foot 304 of the stabilization assembly 300 provides an additional point of contact with the floor surface FS such that the mobile medical system 100 does not tilt or shift relative to the floor surface FS during operation of the mobile medical system 100.

With continued reference to FIGS. 9E through 10F, in one version, the top end 304A of the foot 304 may define a chamfer face CF and the chock 312 may define a wedge face WF configured to abut the chamfer face CF when the chock 312 is in the engaged position 312E to inhibit movement of the foot 304 away from the floor surface FS. Advantageously, the angled profiles of the chamfer face CF and the wedge face WF cooperate to permit the chock 312 to abut the top end 304A of the foot 304 despite moderate vertical position variances of the foot 304 relative to the stabilization housing 302. For example, if the floor surface FS is uneven such that the bottom end 304B of the foot 304 is arranged slightly above or slightly below the contact surface 106, the angled profiles of the chamfer face CF and the wedge face WF cooperate to allow the chock 312 to abut the top end 304A of the foot 304 to inhibit movement of the foot 304 away from the floor surface FS when the retainer 310 is in the brace state 310B. In other words, the angled profiles of the chamfer face CF and the wedge face WF cooperate to allow the chock 312 to abut the top end 304A of the foot 304 when the foot 304 is positioned anywhere beyond the threshold displacement TD to inhibit movement of the foot 304 away from the floor surface FS when the retainer 310 is in the brace state 310B.

The sequence from FIGS. 9F and 10F to 9H and 10H show base lift 110 the moving back from the parked mode PM to the transport mode TM. In other words, the sequence from FIGS. 9F and 10F to 9H and 10H show the base lift 110 raising the base 102 relative to the floor surface FS to lift the contact surface 106 off of the floor surface FS. Here, referring to FIGS. 9G and 10G, as the base lift 110 begins to lift the base 102 relative to the floor surface FS, the foot biasing element 306 urges the foot 304 toward the extended foot position 304E. As a result of the foot 304 moving toward the extended foot position 304E, the post 324 moves within the slot 322 such that the finger lever 320 pivots relative to the stabilization housing 302 to move the finger 318 toward engagement with the chock 312. Still referring to FIGS. 9G and 10G, the finger 318 may deflect around the chock 312 (i.e., toward the retracted position 318R) as the foot 304 returns to the extended foot position 304E. FIGS. 9H and 10H show the base lift 110 reaching the transport mode TM. As a result, the foot biasing element 306 returns the foot 304 to the extended foot position 304E. With continued reference to FIGS. 9H and 10H, the retainer 310 may further include a finger biasing element 328 disposed between the finger 318 and the finger lever 320 to urge the finger 318 back to the deployed position 318D. Thus, the sequence from FIGS. 9G and 10G to 9H and 10H shows the finger 318 returning to the deployed position 318D in response to the foot 304 reaching the extended foot position 304E. Accordingly, the stabilization assembly 300 is reset and ready for another cycle of operation.

It will be further appreciated that the terms “include,” “includes,” and “including” have the same meaning as the terms “comprise,” “comprises,” and “comprising.”

Several embodiments have been discussed in the foregoing description. However, the embodiments discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

The present disclosure also comprises the following clauses, with specific features laid out in dependent clauses, that may specifically be implemented as described in greater detail with reference to the configurations and drawings above.

### CLAUSES

I. A mobile medical system comprising:
a base including:
   a base housing defining a contact surface,
   one or more wheels, and
   a base lift interposed between the base housing and the one or more wheels for moving the contact surface relative to a floor surface, the base lift operable between:
      a parked mode where the contact surface abuts the floor surface to inhibit movement of the base along the floor surface, and
      a transport mode where the contact surface is spaced above the floor surface and with the one or more wheels supporting the base for movement along the floor surface; and
a stabilization assembly for providing an additional point of contact with the floor surface in the parked mode, the stabilization assembly including:
   a stabilization housing coupled to the base,
   a foot extending between a top end and a bottom end and supported for displacement relative to the stabilization housing between a plurality of foot positions including an extended foot position where the bottom end is arranged vertically between the contact surface and the floor surface in the transport mode,
   a foot biasing element operatively attached to the foot to urge the foot towards the extended foot position, and
   a retainer operable between a released state to permit movement of the foot relative to the stabilization housing, and a brace state to inhibit movement of the foot away from the floor surface, the retainer being configured to change operation from the released state to the brace state in response to movement of the foot beyond a threshold displacement from the extended foot position occurring in response to abutment of the foot with the floor surface as the base lift moves from the transport mode towards the parked mode.
II. The mobile medical system of clause I, wherein the retainer further comprises a chock arranged for movement between:
an engaged position where the chock abuts the top end of the foot to inhibit movement of the foot away from the floor surface when the retainer is in the brace state, and
a disengaged position where the chock is spaced from the top end of the foot to permit movement of the foot relative to the stabilization housing when the retainer is in the released state.
III. The mobile medical system of clause II, wherein the top end of the foot defines a chamfer face, and the chock defines a wedge face configured to abut the chamfer face when the chock is in the engaged position to inhibit movement of the foot away from the floor surface.
IV. The mobile medical system of any of clauses II-III wherein the retainer further comprises a retainer biasing element disposed in the stabilization housing and operatively attached to the chock to urge the chock to the engaged position.
V. The mobile medical system of clause IV, wherein the retainer further comprises a damper configured to slow translation of the chock from the disengaged position to the engaged position.
VI. The mobile medical system of clause V, wherein the retainer further comprises a finger operatively attached to the foot, the finger configured to engage the chock when the foot is in the extended foot position and configured to displace the chock toward the disengaged position as the foot displaces from the extended foot position in response to abutment of the foot with the floor surface.
VII. The mobile medical system of clause VI, wherein the finger is configured to disengage from the chock as the foot reaches the threshold displacement from the extended foot position such that the retainer biasing element urges the chock to the engaged position to bring the chock into abutment with the top end of the foot to inhibit movement of the foot away from the floor surface.
VIII. The mobile medical system of clause VII, wherein the retainer further comprises a finger lever pivotably attached to the stabilization housing and supporting the finger, wherein the finger lever defines a slot, and the foot includes a post disposed in the slot; and
wherein movement of the post within the slot moves the finger to displace the chock toward the disengaged position as the foot displaces from the extended foot position in response to abutment of the foot with the floor surface.
IX. The mobile medical system of clause VIII, wherein the finger is attached to the finger lever for pivoting movement relative to the finger lever between:
a deployed position where the finger is arranged to engage the chock when the foot is in the extended foot position to displace the chock toward the disengaged position as the foot displaces from the extended foot position in response to abutment of the foot with the floor surface, and
a retracted position where the finger pivots relative to the finger lever in response to the foot reaching the threshold displacement from the extended foot position such that the finger disengages from the chock and the chock translates toward the engaged position to inhibit movement of the foot away from the floor surface.
X. The mobile medical system of clause IX, wherein the retainer further comprises a finger biasing element disposed between the finger and the finger lever and configured to urge the finger toward the deployed position such that the finger returns to the deployed position in response to the base lift moving to the transport mode.
XI. The mobile medical system of any of clauses I-X, further comprising one or more casters each including one of the wheels, each of the one or more casters supported by a pivoting caster arm assembly interposed between the base and the caster,
wherein each pivoting caster arm assembly is configured to pivot relative to the base to move each caster between:
   a retracted position where each caster is spaced from the base at a first offset distance when the base lift is in the parked mode to permit the contact surface to abut the floor surface to inhibit movement of the base along the floor surface, and
an extended position where each caster is spaced from the base at a second offset distance, greater than the first offset distance, when the base lift is in the transport mode to lift the base relative to the floor surface such that the contact surface is spaced above the floor surface and the one or more wheels support the base for movement along the floor surface.
   XII. The mobile medical system of any of clauses I-XI, wherein the base housing supports an imaging gantry for acquiring image data of a patient.
   XIII. The mobile medical system of clause XII, wherein the imaging gantry including at least one imaging component and defines an imaging bore.
   XIV. The mobile medical system of clause XIII, wherein the at least one imaging component includes a rotor supporting an x-ray source and a detector and disposed within a gantry housing defined by the imaging gantry for rotation around the imaging bore.
   XV. The mobile medical system of clause XIV, wherein the x-ray source includes a fan-beam x-ray source, and the detector includes an array of detectors.
   XVI. The mobile medical system of any of clauses XIII-XV, further comprising a pedestal mounted to the base and configured to support a patient support above the base and within the imaging bore.
   XVII. The mobile medical system of any of clauses XIII-XVI, wherein the base defines a track extending between a first track end and a second track end.
   XVIII. The mobile medical system of clause XVII, further comprising a gantry mount disposed between the base and the imaging gantry for supporting the imaging gantry for movement along the track between a plurality of track poses including a park pose defined with the gantry mount arranged adjacent to the first track end.
   XIX. The mobile medical system of clause XVIII, further comprising a translation mechanism interposed between the base and the gantry mount to drive the gantry mount between the plurality of track poses in an imaging mode to acquire image data of a patient within the imaging bore.
   XX. The mobile medical system of clause XIX, wherein:
   the at least one imaging component includes a rotor supporting an x-ray source and a detector and disposed within a gantry housing defined by the imaging gantry for rotation around the imaging bore; and
   the rotor rotates around the imaging bore as the translation mechanism drives the gantry mount along the track in the imaging mode to acquire helical scan x-ray CT images of a patient within the imaging bore.
   XXI. The mobile medical system of any of clauses XIX-XX, further comprising:
   a translation motor operatively attached to the translation mechanism to drive the gantry mount between the plurality of track poses; and
   a controller in communication with the translation motor to control operation of the translation motor.
   XXII. The mobile medical system of any of clauses XIX-XXI, wherein the gantry mount includes:
   a gantry mount base operatively attached to the base,
   a gantry mount member operatively attached to the gantry mount base for rotation relative to the gantry mount base, the gantry mount member supporting the imaging gantry such that the gantry mount member and the imaging gantry are configured to rotate together about a first axis relative to the base.
   XXIII. The mobile medical system of clause XXII, wherein:
   the imaging bore defines an imaging axis that is parallel to the track where the gantry mount is in the park pose and the mobile medical system is in the imaging mode, and
   the plurality of track poses of the gantry mount includes a transport pose where the gantry mount is arranged between the first track end and the second track end, and the gantry mount member and the imaging gantry are rotated such that the imaging axis is transverse to the track.
   XXIV. The mobile medical system of any of clauses XXII-XXIII, wherein the gantry mount member includes a gimbal having a pair of arms, each arm coupled to an opposite side of the imaging gantry to support the imaging gantry above the base and the gimbal, wherein the imaging gantry is configured to tilt about a second axis relative to the gimbal.
   XXV. The mobile medical system of any of clauses XXII-XXIV, further comprising:
   a gantry motor interposed between the gantry mount base and the gantry mount member for rotating the gantry mount member relative to the base about the first axis; and
   a controller in communication with the gantry motor to control operation of the gantry motor.
   XXVI. The mobile medical system of any of clauses XII-XXV, further comprising a robotic arm extending between a base end operatively attached to the imaging gantry and a mount end arranged for movement relative to the base end.
   XXVII. The mobile medical system of clause XXVI, further comprising an end effector attached to the mount end of the robotic arm and configured to support a tool for engaging a target site.
   XXVIII. The mobile medical system of clause XXVII, wherein the robotic arm is configured to maintain alignment of the tool relative to the target site.
   XXIX. A mobile medical imaging system comprising:
   an imaging gantry having at least one imaging component for acquiring image data of a patient;
   a base including:
      a base housing supporting the imaging gantry and defining a contact surface,
      one or more wheels, and
      a base lift interposed between the base housing and the one or more wheels for moving the contact surface relative to a floor surface, the base lift operable between:
         a parked mode where the contact surface abuts the floor surface to inhibit movement of the base along the floor surface, and
         a transport mode where the contact surface is spaced above the floor surface and the one or more wheels support the base for movement along the floor surface; and
   a stabilization assembly for providing an additional point of contact with the floor surface in the parked mode, the stabilization assembly including:
      a stabilization housing coupled to the base,
      a foot extending between a top end and a bottom end and supported for displacement relative to the stabilization housing between a plurality of foot positions including an extended foot position where the bottom end is arranged vertically between the contact surface and the floor surface in the transport mode,
      a biasing element operatively attached to the foot to urge the foot towards the extended foot position, and
      a retainer operable between a released state to permit movement of the foot relative to the stabilization housing, and brace state to inhibit movement of the foot away from the floor surface, the retainer being configured to change operation from the released state to the brace state in response to movement of the foot beyond a threshold displacement from the extended foot position occurring in response to abutment of the foot with the floor surface as the base lift moves from the transport mode towards the parked mode.
   XXX. The mobile medical imaging system of clause XXIX, wherein the retainer further comprises a chock arranged for movement between:
   an engaged position where the chock abuts the top end of the foot to inhibit movement of the foot away from the floor surface when the retainer is in the brace state, and
   a disengaged position where the chock is spaced from the top end of the foot to permit movement of the foot relative to the stabilization housing when the retainer is in the released state.
   XXXI. The mobile medical imaging system of clause XXX, wherein the top end of the foot defines a chamfer face, and the chock defines a wedge face configured to abut the chamfer face when the chock is in the engaged position to inhibit movement of the foot away from the floor surface.
   XXXII. The mobile medical imaging system of any of clauses XXX-XXXI, wherein the retainer further comprises a retainer biasing element disposed in the stabilization housing and operatively attached to the chock to urge the chock to the engaged position.
   XXXIII. The mobile medical imaging system of clause XXXII, wherein the retainer further comprises a damper configured to slow translation of the chock from the disengaged position to the engaged position.
   XXXIV. The mobile medical imaging system of clause XXXIII, wherein the retainer further comprises a finger operatively attached to the foot, the finger configured to engage the chock when the foot is in the extended foot position and configured to displace the chock toward the disengaged position as the foot displaces from the extended foot position in response to abutment of the foot with the floor surface.
   XXXV. The mobile medical imaging system of clause XXXIV, wherein the finger is configured to disengage from the chock as the foot reaches the threshold displacement from the extended foot position such that the retainer biasing element urges the chock to the engaged position such that the chock abuts the top end of the foot to inhibit movement of the foot away from the floor surface.
   XXXVI. The mobile medical imaging system of clause XXXV, wherein the retainer further comprises a finger lever pivotable attached to the stabilization housing and supporting the finger, wherein the finger lever defines a slot, and the foot includes a post disposed in the slot; and
   wherein movement of the post within the slot moves the finger to displace the chock toward the disengaged position as the foot displaces from the extended foot position in response to abutment of the foot with the floor surface.
   XXXVII. The mobile medical imaging system of clause XXXVI, wherein the finger is attached to the finger lever for pivoting movement relative to the finger lever between:
   a deployed position where the finger is arranged to engage the chock when the foot is in the extended foot position to displace the chock toward the disengaged position as the foot displaces from the extended foot position in response to abutment of the foot with the floor surface, and
   a retracted position where the finger pivots relative to the finger lever in response to the foot reaching the threshold displacement from the extended foot position such that the finger disengages from the chock and the chock translates toward the engaged position to inhibit movement of the foot away from the floor surface.
   XXXVIII. The mobile medical imaging system of clause XXXVII, wherein the retainer further comprises a finger biasing element disposed between the finger and the finger lever and configured to urge the finger toward the deployed position such that the finger returns to the deployed position in response to the base lift moving to the transport mode.
   XXXIX. The mobile medical imaging system of any of clauses IX-XXXVIII, further comprising one or more casters each including one of the wheels each of the one or more casters supported by a pivoting caster arm assembly interposed between the base and the caster,
   wherein each pivoting caster arm assembly is configured to pivot relative to the base to move each caster between:
      a retracted position where each caster is spaced from the base at a first offset distance when the base lift is in the parked mode to permit the contact surface to abut the floor surface to inhibit movement of the base along the floor surface, and
an extended position where each caster is spaced from the base at a second offset distance, greater than the first offset distance, when the base lift is in the transport mode to lift the base relative to the floor surface such that the contact surface is spaced above the floor surface and the one or more wheels support the base for movement along the floor surface.
   XL. The mobile medical imaging system of any of clauses XXIX-XXXIX, wherein the at least one imaging component includes a rotor supporting an x-ray source and a detector and disposed within a gantry housing defined by the imaging gantry for rotation around an imaging bore.
   XLI. The mobile medical imaging system of clause XL, wherein the x-ray source includes a fan-beam x-ray source, and the detector includes an array of detectors.
   XLII. The mobile medical imaging system of any of clauses XL-XLI, further comprising a pedestal mounted to the base and configured to support a patient support above the base and within the imaging bore.
   XLIII. The mobile medical imaging system of any of clauses XL-XLII, wherein the base defines a track extending between a first track end and a second track end.
   XLIV. The mobile medical imaging system of clause XLIII, further comprising a gantry mount disposed between the base and the imaging gantry for supporting the imaging gantry for movement along the track between a plurality of track poses including a park pose defined with the gantry mount arranged adjacent to the first track end.
   XLV. The mobile medical imaging system of clause XLIV, further comprising a translation mechanism interposed between the base and the gantry mount to drive the gantry mount between the plurality of track poses in an imaging mode to acquire image data of a patient within the imaging bore.
   XLVI. The mobile medical imaging system of clause XLV, wherein:
   the at least one imaging component includes a rotor supporting an x-ray source and a detector and disposed within a gantry housing defined by the imaging gantry for rotation around the imaging bore; and
   the rotor rotates around the imaging bore as the translation mechanism drives the gantry mount along the track in the imaging mode to acquire helical scan x-ray CT images of a patient within the imaging bore.
   XLVII. The mobile medical imaging system of any of clauses XLV-XLVI, further comprising:
   a translation motor operatively attached to the translation mechanism to drive the gantry mount between the plurality of track poses; and
   a controller in communication with the translation motor to control operation of the translation motor.
   XLVIII. The mobile medical imaging system of any of clauses XLV-XLVII, wherein the gantry mount includes:
   a gantry mount base operatively attached to the base,
   a gantry mount member operatively attached to the gantry mount base for rotation relative to the gantry mount base, the gantry mount member supporting the imaging gantry such that the gantry mount member and the imaging gantry are configured to rotate together about a first axis relative to the base.
   XLIX. The mobile medical imaging system of clause XLVIII, wherein:
   the imaging bore defines an imaging axis that is parallel to the track where the gantry mount is in the park pose and the mobile medical imaging system is in the imaging mode, and
   the plurality of track poses of the gantry mount includes a transport pose where the gantry mount is arranged between the first track end and the second track end, and the gantry mount member and the imaging gantry are rotated such that the imaging axis is transverse to the track.
   L. The mobile medical imaging system of clause XLIX, wherein the gantry mount member includes a gimbal having a pair of arms, each arm coupled to an opposite side of the imaging gantry to support the imaging gantry above the base and the gimbal, wherein the imaging gantry is configured to tilt about a second axis relative to the gimbal.
   LI. The mobile medical imaging system of any of clauses XLIX-L, further comprising:
   a gantry motor interposed between the gantry mount base and the gantry mount member for rotating the gantry mount member relative to the base about the first axis; and
   a controller in communication with the gantry motor to control operation of the gantry motor.
   LII. The mobile medical imaging system of any of clauses XXIX-LI, further comprising a robotic arm extending between a base end operatively attached to the imaging gantry and a mount end arranged for movement relative to the base end.
   LIII. The mobile medical imaging system of clause LII, further comprising an end effector attached to the mount end of the robotic arm and configured to support a tool for engaging a target site.
   LIV. The mobile medical imaging system of clause LIII, wherein the robotic arm is configured to maintain alignment of the tool relative to the target site.
   LV. A stabilization assembly configured to be coupled to a mobile medical system for providing an additional point of contact with a floor surface, the stabilization assembly including:
   a stabilization housing configured to be coupled to the mobile medical system;
   a foot extending between a top end and a bottom end and supported for displacement relative to the stabilization housing between a plurality of foot positions including an extended foot position where the bottom end extends from the stabilization housing at a maximum distance;
   a biasing element operatively attached to the foot to urge the foot towards the extended foot position; and
   a retainer operable between a released state to permit movement of the foot relative to the stabilization housing, and brace state to inhibit movement of the foot away from the floor surface, the retainer being configured to change operation from the released state to the brace state in response to movement of the foot beyond a threshold displacement from the extended foot position occurring in response to abutment of the foot with the floor surface.
   LVI. The stabilization assembly of clause LV, wherein the retainer further comprises a chock arranged for movement between:
   an engaged position where the chock abuts the top end of the foot to inhibit movement of the foot away from the floor surface when the retainer is in the brace state, and
   a disengaged position where the chock is spaced from the top end of the foot to permit movement of the foot relative to the stabilization housing when the retainer is in the released state.
   LVII. The stabilization assembly of clause LVI, wherein the top end of the foot defines a chamfer face, and the chock defines a wedge face configured to abut the chamfer face when the chock is in the engaged position to inhibit movement of the foot away from the floor surface.
   LVIII. The stabilization assembly of any of clauses LVI-LVII, wherein the retainer further comprises a retainer biasing element disposed in the stabilization housing and operatively attached to the chock to urge the chock to the engaged position.
   LIX. The stabilization assembly of clause LVIII, wherein the retainer further comprises a damper configured to slow translation of the chock from the disengaged position to the engaged position.
   LX. The stabilization assembly of clause LIX, wherein the retainer further comprises a finger operatively attached to the foot, the finger configured to engage the chock when the foot is in the extended foot position and configured to displace the chock toward the disengaged position as the foot displaces from the extended foot position in response to abutment of the foot with the floor surface.
   LXI. The stabilization assembly of clause LX, wherein the finger is configured to disengage from the chock as the foot reaches the threshold displacement from the extended foot position such that the retainer biasing element urges the chock to the engaged position to bring the chock into abutment with the top end of the foot to inhibit movement of the foot away from the floor surface.
   LXII. The stabilization assembly of clause LXI, wherein the retainer further comprises a finger lever pivotable attached to the stabilization housing and supporting the finger, wherein the finger lever defines a slot, and the foot includes a post disposed in the slot; and
   wherein movement of the post within the slot moves the finger to displace the chock toward the disengaged position as the foot displaces from the extended foot position in response to abutment of the foot with the floor surface.
   LXIII. The stabilization assembly of clause LXII, wherein the finger is attached to the finger lever for pivoting movement relative to the finger lever between:
   a deployed position where the finger is arranged to engage the chock when the foot is in the extended foot position to displace the chock toward the disengaged position as the foot displaces from the extended foot position in response to abutment of the foot with the floor surface, and
   a retracted position where the finger pivots relative to the finger lever in response to the foot reaching the threshold displacement from the extended foot position such that the finger disengages from the chock and the chock translates toward the engaged position to inhibit movement of the foot away from the floor surface.
   LXIV. The stabilization assembly of clause LXIII, wherein the retainer further comprises a finger biasing element disposed between the finger and the finger lever and configured to urge the finger toward the deployed position such that the finger returns to the deployed position in response to the foot moving toward the extended foot position.

1. A mobile medical system comprising:
a base including:
a base housing defining a contact surface,
one or more wheels, and
a base lift interposed between the base housing and the one or more wheels for moving the contact surface relative to a floor surface, the base lift operable between:
a parked mode where the contact surface abuts the floor surface to inhibit movement of the base along the floor surface, and
a transport mode where the contact surface is spaced above the floor surface and with the one or more wheels supporting the base for movement along the floor surface; and
a stabilization assembly for providing an additional point of contact with the floor surface in the parked mode, the stabilization assembly including:
a stabilization housing coupled to the base,
a foot extending between a top end and a bottom end and supported for displacement relative to the stabilization housing between a plurality of foot positions including an extended foot position where the bottom end is arranged vertically between the contact surface and the floor surface in the transport mode,
a foot biasing element operatively attached to the foot to urge the foot towards the extended foot position, and
a retainer operable between a released state to permit movement of the foot relative to the stabilization housing, and a brace state to inhibit movement of the foot away from the floor surface, the retainer being configured to change operation from the released state to the brace state in response to movement of the foot beyond a threshold displacement from the extended foot position occurring in response to abutment of the foot with the floor surface as the base lift moves from the transport mode towards the parked mode.

2. The mobile medical system of claim 1, wherein the retainer further comprises a chock arranged for movement between:
an engaged position where the chock abuts the top end of the foot to inhibit movement of the foot away from the floor surface when the retainer is in the brace state, and
a disengaged position where the chock is spaced from the top end of the foot to permit movement of the foot relative to the stabilization housing when the retainer is in the released state.

3. The mobile medical system of claim 2, wherein the top end of the foot defines a chamfer face, and the chock defines a wedge face configured to abut the chamfer face when the chock is in the engaged position to inhibit movement of the foot away from the floor surface.

4. The mobile medical system of claim 2, wherein the retainer further comprises:
a retainer biasing element disposed in the stabilization housing and operatively attached to the chock to urge the chock to the engaged position,
a damper configured to slow translation of the chock from the disengaged position to the engaged position, and
a finger operatively attached to the foot, the finger configured to engage the chock when the foot is in the extended foot position and configured to displace the chock toward the disengaged position as the foot displaces from the extended foot position in response to abutment of the foot with the floor surface.

5. (canceled)

6. (canceled)

7. The mobile medical system of claim 4, wherein the finger is configured to disengage from the chock as the foot reaches the threshold displacement from the extended foot position such that the retainer biasing element urges the chock to the engaged position to bring the chock into abutment with the top end of the foot to inhibit movement of the foot away from the floor surface
wherein the retainer further comprises a finger lever pivotably attached to the stabilization housing and supporting the finger, wherein the finger lever defines a slot, and the foot includes a post disposed in the slot; and
wherein movement of the post within the slot moves the finger to displace the chock toward the disengaged position as the foot displaces from the extended foot position in response to abutment of the foot with the floor surface.

8. (canceled)

9. The mobile medical system of claim 7, wherein the finger is attached to the finger lever for pivoting movement relative to the finger lever between:
a deployed position where the finger is arranged to engage the chock when the foot is in the extended foot position to displace the chock toward the disengaged position as the foot displaces from the extended foot position in response to abutment of the foot with the floor surface, and
a retracted position where the finger pivots relative to the finger lever in response to the foot reaching the threshold displacement from the extended foot position such that the finger disengages from the chock and the chock translates toward the engaged position to inhibit movement of the foot away from the floor surface; and
wherein the retainer further comprises a finger biasing element disposed between the finger and the finger lever and configured to urge the finger toward the deployed position such that the finger returns to the deployed position in response to the base lift moving to the transport mode.

10. (canceled)

11. The mobile medical system of claim 1, further comprising one or more casters each including one of the wheels, each of the one or more casters supported by a pivoting caster arm assembly interposed between the base and the caster,
wherein each pivoting caster arm assembly is configured to pivot relative to the base to move each caster between:
a retracted position where each caster is spaced from the base at a first offset distance when the base lift is in the parked mode to permit the contact surface to abut the floor surface to inhibit movement of the base along the floor surface, and
an extended position where each caster is spaced from the base at a second offset distance, greater than the first offset distance, when the base lift is in the transport mode to lift the base relative to the floor surface such that the contact surface is spaced above the floor surface and the one or more wheels support the base for movement along the floor surface.

12. (canceled)

13. The mobile medical system of claim 1, wherein the base housing supports an imaging gantry for acquiring image data of a patient, the imaging gantry including at least one imaging component and defines an imaging bore.

14. The mobile medical system of claim 13, wherein the at least one imaging component includes a rotor supporting an x-ray source and a detector and disposed within a gantry housing defined by the imaging gantry for rotation around the imaging bore; and
wherein the x-ray source includes a fan-beam x-ray source, and the detector includes an array of detectors.

15. (canceled)

16. The mobile medical system of claim 13, further comprising a pedestal mounted to the base and configured to support a patient support above the base and within the imaging bore.

17. The mobile medical system of claim 13, wherein the base defines a track extending between a first track end and a second track end.

18. The mobile medical system of claim 17, further comprising a gantry mount disposed between the base and the imaging gantry for supporting the imaging gantry for movement along the track between a plurality of track poses including a park pose defined with the gantry mount arranged adjacent to the first track end.

19. The mobile medical system of claim 18, further comprising a translation mechanism interposed between the base and the gantry mount to drive the gantry mount between the plurality of track poses in an imaging mode to acquire image data of a patient within the imaging bore.

20. The mobile medical system of claim 19, wherein:
the at least one imaging component includes a rotor supporting an x-ray source and a detector and disposed within a gantry housing defined by the imaging gantry for rotation around the imaging bore; and
the rotor rotates around the imaging bore as the translation mechanism drives the gantry mount along the track in the imaging mode to acquire helical scan x-ray CT images of a patient within the imaging bore.

21. The mobile medical system of claim 19, further comprising:
a translation motor operatively attached to the translation mechanism to drive the gantry mount between the plurality of track poses; and
a controller in communication with the translation motor to control operation of the translation motor.

22. The mobile medical system of claim 19, wherein the gantry mount includes:
a gantry mount base operatively attached to the base,
a gantry mount member operatively attached to the gantry mount base for rotation relative to the gantry mount base, the gantry mount member supporting the imaging gantry such that the gantry mount member and the imaging gantry are configured to rotate together about a first axis relative to the base.

23. The mobile medical system of claim 22, wherein:
the imaging bore defines an imaging axis that is parallel to the track where the gantry mount is in the park pose and the mobile medical system is in the imaging mode, and
the plurality of track poses of the gantry mount includes a transport pose where the gantry mount is arranged between the first track end and the second track end, and the gantry mount member and the imaging gantry are rotated such that the imaging axis is transverse to the track.

24. The mobile medical system of claim 22, wherein the gantry mount member includes a gimbal having a pair of arms, each arm coupled to an opposite side of the imaging gantry to support the imaging gantry above the base and the gimbal, wherein the imaging gantry is configured to tilt about a second axis relative to the gimbal.

25. The mobile medical system of claim 22, further comprising:
a gantry motor interposed between the gantry mount base and the gantry mount member for rotating the gantry mount member relative to the base about the first axis; and
a controller in communication with the gantry motor to control operation of the gantry motor.

26. The mobile medical system of claim 1, wherein the base housing supports an imaging gantry for acquiring image data of a patient; and further comprising:
a robotic arm extending between a base end operatively attached to the imaging gantry and a mount end arranged for movement relative to the base end; and
an end effector attached to the mount end of the robotic arm and configured to support a tool for engaging a target site, wherein the robotic arm is configured to maintain alignment of the tool relative to the target site.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)
